(19) United States
(12) Patent Application Publication
(10) Pub. No.: US 2024/0277256 A1
(43) Pub. Date: Aug. 22, 2024

(54) FORCE MEASUREMENT PLATFORM FOR DETERMINING AND MONITORING POSTURAL STABILITY

(71) Applicant: Imam Abdulrahman Bin Faisal University, Dammam (SA)

(72) Inventors: Ibrahim ALJAMAAN, Dammam (SA); Mariam HEGAZI, Dammam (SA); Heba ALWALILY, Dammam (SA); Reem ALSAIHATI, Dammam (SA); Shahad ALDOSSARY, Dammam (SA); Shatha ALAJMAH, Dammam (SA); Tahani ALNAJJAR, Dammam (SA)

(73) Assignee: Imam Abdulrahman Bin Faisal University, Dammam (SA)

(21) Appl. No.: 18/168,964

(22) Filed: Feb. 14, 2023

(51) Int. Cl. A61B 5/103; A61B 5/00; A61B 5/11

(52) U.S. Cl. CPC A61B 5/1036; A61B 5/112; A61B 5/4023; G01L 1/04; G01L 5/16

(57) ABSTRACT

A force measurement platform is described. The force measurement platform includes a force measurement assembly comprising surface plate having first side and second side opposite to first side, and base plate having first side and second side opposite to the first side. The force measurement platform includes plurality of force sensors, where the force sensors are mutually diagonally arranged in four quadrants of the force measurement assembly with each quadrant containing at least one force sensor. The force measurement platform includes an optional secondary force sensor located in a geometric center of the force measurement assembly. The force measurement platform includes a data acquisition and processing unit comprising a plurality of signal conditioners and a microcontroller, and a computing device, where the computing device has a communications interface coupled to the data acquisition and processing unit, and where the force measurement platform removably retains the computing device.

### STATEMENT OF ACKNOWLEDGEMENT

This research was supported by the Deanship of Scientific Research at Imam Abdulrahman Bin Faisal University under the project 2021-143-Eng.

### BACKGROUND

### Technical Field

The present disclosure is directed to a force measurement platform for determining and monitoring postural stability of patients.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Postural stability is necessary for maintaining balance while performing tasks and reacting to unanticipated disturbances in daily life. The continuous monitoring and evaluation of postural stability are essential in maintaining balance. It also helps in the early detection of defects and abnormalities related to balance, which, if not treated, may increase the risk of falling during standing or walking, leading to injuries and disorders, such as lower-limb injuries [S. Golriz, J. J. Hebert, K. B. Foreman, and B. F. Walker, “The validity of a portable clinical force plate in assessment of static postural control: concurrent validity study,” Chiropr. Man. Ther., vol. 20, no. 1, pp. 1-6, May 2012]. In an example, monitoring and evaluation of postural stability may be used to identify patients at high risk of injury. The recovery process from these injuries usually takes a long time and may be cost-intensive [N. Millikan, D. R. Grooms, B. Hoffman, and J. E. Simon, “The Development and Reliability of 4 Clinical Neurocognitive Single-Leg Hop Tests: Implications for Return to Activity Decision-Making.,” undefined, vol. 28, no. 5, pp. 1-9, July 2019].

In the past, systems and methods have been developed for evaluating postural abnormalities. Visual observation and plumb line methods are examples of primitive methods used by physiotherapist. The visual observation method does not require the use of any equipment. However, quantitative data cannot be gathered using these systems and methods. As a result, minor postural deviations are undetectable. A plumb line together with a postural grid is developed for evaluating postural abnormalities, however these systems and methods suffer from various limitations including high cost and inaccurate detection of postural stability [D. Singla and Z. Veqar, “Methods of Postural Assessment Used for Sports Persons,” J. Clin. Diagn. Res., vol. 8, no. 4, p. LE01, 2014].

A force plate is designed and developed to measure the changes in voltage which is proportional to the force applied. This is achieved by using one of two types of sensors to measure the ground reaction force (GRF) including load cells and piezoelectric sensors. The orientation of sensors helps in translating the magnitude and direction of forces which is GRF into electrical signals [K. A. Lamkin-Kennard and M. B. Popovic, “Sensors: Natural and Synthetic Sensors,” Biomechatronics, pp. 81-107, January 2019; R. E. Nordquist, E. Meijer, F. J. van der Staay, and S. S. Arndt, “Pigs as Model Species to Investigate Effects of Early Life Events on Later Behavioral and Neurological Functions,” Anim. Model. Study Hum. Dis. Second Ed., pp. 1003-1030, January 2017].

In view of the forgoing, one objective of the present disclosure is to describe a force measurement platform. Furthermore, a second objective of the present disclosure is to present a method for determining and monitoring postural stability with acceptable quality and at a low cost.

### SUMMARY

In an exemplary embodiment, a force measurement platform is disclosed. The force measurement platform contains a force measurement assembly. The force measurement assembly includes a surface plate having a first side and a second side opposite to the first side, and a base plate having a first side and a second side opposite to the first side, where the second side of the surface plate is above and opposite to the first side of the base plate. The force measurement platform also includes a plurality of force sensors. The plurality of force sensors are mutually diagonally arranged in four quadrants of the force measurement assembly with each quadrant containing at least one force sensor. The force measurement platform includes an optional secondary force sensor located in a geometric center of the force measurement assembly. The surface plate is supported on the base plate via the plurality of force sensors and the optional secondary force sensor. The force measurement platform includes a data acquisition and processing unit. The data acquisition and processing unit includes a plurality of signal conditioners and a microcontroller. The force measurement platform also includes a computing device. The computing device has a communications interface coupled to the data acquisition and processing unit. The force measurement platform removably retains the computing device.

In some embodiments, the force sensor of the force measurement platform includes a beam portion having a first end and a second end. In some embodiments, the force sensor further includes a plurality of deformation sensing elements disposed on the beam portion of the force sensor along a longitudinal axis between the first end and the second end of the beam portion, in which the plurality of deformation sensing elements can covert an input mechanical load to an electrical signal representative of the load that is applied to the surface plate. In some further embodiments, the first end of the beam portion is connected to the second side of the surface plate around at least one first pivot axis that is perpendicular to the surface plate, and is configured to receive at least a portion of a load that is applied to the surface plate. In some further preferred embodiments, the second end of the beam portion is mounted to the first side of the base plate around at least one second pivot axis that is perpendicular to the base plate. In some more preferred embodiments, the at least one first pivot axis is parallel to the at least one second pivot axis.

In some embodiments, the optional secondary force sensor of the force measurement platform includes a beam portion having a first end and a second end. In some embodiments, the optional secondary force sensor includes a plurality of deformation sensing elements disposed on the beam portion of the force sensor along a longitudinal axis between the first end and the second end of the beam portion, in which the plurality of deformation sensing elements can covert an input mechanical load to an electrical signal representative of the load that is applied to the surface plate. In some further embodiments, the first end of the beam portion is connected to the second side of the surface plate around at least one first pivot axis that is perpendicular to the surface plate, and is configured to receive at least a portion of a load that is applied to the surface plate. In some further preferred embodiments, the second end of the beam portion is mounted to the first side of the base plate around at least one second pivot axis that is perpendicular to the base plate. In some more preferred embodiments, the at least one first pivot axis is parallel to the at least one second pivot axis.

In some embodiments, the force sensor is at least one selected from the group consisting of a capacitive displacement sensor, an inductive force sensor, a strain gauge, a piezoelectric force sensor, a force sensing resistor, a piezoresistive force sensor, a thin film force sensor, and a quantum tunnelling composite-based force sensor.

In some embodiments, the optional secondary force sensor is at least one selected from the group consisting of a capacitive displacement sensor, an inductive force sensor, a strain gauge, a piezoelectric force sensor, a force sensing resistor, a piezoresistive force sensor, a thin film force sensor, and a quantum tunnelling composite-based force sensor.

In some embodiments, each of the plurality of signal conditioners is operatively connected to each of the plurality of force sensors and the optional secondary force sensor of the force measurement assembly. In some further embodiments, the plurality of signal conditioners are operatively connected to the microcontroller.

In some embodiments, the plurality of signal conditioners contains at least one amplifier, at least one high-pass filter, and at least one low-pass filter.

In some embodiments, wherein the microcontroller contains a memory for saving or installing an application program, or a software program, in which the application program or the software program from an internet or a cloud server is downloaded for interpreting and executing digital signals.

In some embodiments, the data acquisition and processing unit is configured to receive one or more signals that are representative of a load being applied to a surface of the force measurement assembly and to record a location of the applied load on the surface of the force measurement assembly by computing a center of pressure (COP) for the applied load as determined by ASTM F3109-16.

In some embodiments, the data acquisition and processing unit is further configured to monitor the signals of the applied load over time to visualize a sway or a change in location of the load being applied on surface of the force measurement assembly by computing the COP.

In some embodiments, the computing device of the force measurement platform presents a data visualization configuration interface according to a load applied to the force measurement platform. In some further embodiments, the computing device includes a personal computer (PC), a server, a mobile computing device, and a computing circuit.

In some embodiments, both the surface plate and the base plate are made of an aluminum alloy that is composed of, in % by mass, less than or equal to 0.4% of iron, less than or equal to 0.25% of silicon, and the balance being aluminum and unavoidable impurities.

In some embodiments, the surface plate is made of stainless steel, brass, heavy duty polycarbonate plastic, and ultra-high molecular weight polyethylene plastic.

In some embodiments, the base plate is made of stainless steel, brass, heavy duty polycarbonate plastic, and ultra-high molecular weight polyethylene plastic.

In some embodiments, the force measurement platform meets the ANSI B157.1 standards.

In some embodiments, the force measurement platform has a load capacity of up to 10 kilonewtons (kN).

In another exemplary embodiment, a method of determining and monitoring postural stability is discloses. The method involves calibrating a force measurement platform. The force measurement platform includes a force measurement assembly. The method includes applying a load to the surface of the force measurement assembly via at least one portion of a body and running the force measurement platform for 10 to 30 seconds to calculate a COP and record a sway. The method further includes comparing the results of COP and sway against a reference database to determine and monitor postural stability.

In some embodiments, the method of determining and monitoring postural stability is conducted in a state selected from the group consisting of eyes open, and eyes closed.

In some embodiments, the calibrating contains removing any loads on the force measurement assembly and zeroizing the readings of the platform. In some further embodiments, the calibrating further includes placing a known mass on the force measurement assembly, running the platform, and recording a displayed mass. In some further preferred embodiments, the calibrating involves calculating a calibration factor via a mathematical formula and setting the calibration factor in the data acquisition and processing unit.

In some embodiments, the mathematical formula (I) is:

$$Calibration factor [ I ] = \frac{Capacity ( g )}{Sensitivity ( \frac{mV}{V} ) \times Excitation Voltage ( V )}$$

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 depicts a block diagram of a force measurement platform, according to aspects of the present disclosure.

FIG. 2A-FIG. 2C illustrate isometric views of a force measurement assembly, according to aspects of the present disclosure.

FIG. 3A-FIG. 3C illustrate wireframe isometric views of the force measurement assembly, according to aspects of the present disclosure.

FIG. 4A shows a top view of the force measurement assembly, according to aspects of the present disclosure.

FIG. 4B shows the force measurement assembly comprising force sensors, according to aspects of the present disclosure.

FIG. 5 shows a side view of the force measurement assembly, according to aspects of the present disclosure.

FIG. 6 shows another isometric view of the force measurement assembly, according to aspects of the present disclosure.

FIG. 7 shows a close-up view of the force measurement assembly, according to aspects of the present disclosure.

FIG. 8 shows yet another isometric view of the force measurement assembly 800, according to aspects of the present disclosure.

FIG. 9 describes a loadcell TAL220 sensor, according to aspects of the present disclosure.

FIG. 10 describes an example of load cell dimensions, according to aspects of the present disclosure.

FIG. 11 depicts a HX711 load cell amplifier, according to aspects of the present disclosure.

FIG. 12 depicts electrical connections of the force measurement platform, according to aspects of the present disclosure.

FIG. 13A-FIG. 13G show different sections of a block diagram of a LabVIEW software program, according to aspects of the present disclosure.

FIG. 14 shows a front panel of a LabVIEW software program, according to aspects of the present disclosure.

FIG. 15A-FIG. 15D show different tabs of the front panel of the LabVIEW software program, according to aspects of the present disclosure.

FIG. 16 illustrates a flowchart of the LabVIEW software program, according to aspects of the present disclosure.

FIG. 17 shows a detailed block diagram of the force measurement platform, according to some aspects.

FIG. 18 describes a manufacturing process of the force measurement platform, according to aspects of the present disclosure.

FIG. 19 shows an example of load cell adjustment, according to aspects of the present disclosure.

FIG. 20 illustrates an example of connections from a load cell to HX711 amplifier, according to aspects of the present disclosure.

FIG. 21 illustrates an example of connections between HX711 amplifiers and Arduino, according to aspects of the present disclosure.

FIG. 22 describes an example of a designed sticker for the force measurement assembly, according to aspects of the present disclosure.

FIG. 23 describes a calibration process, according to aspects of the present disclosure.

FIG. 24 describes an experiment setup with a subject, according to aspects of the present disclosure.

FIG. 25 shows an experiment setup showing sway parameters, according to aspects of the present disclosure.

FIG. 26 shows an example of a normative database, according to aspects of the present disclosure.

FIG. 27 shows a front panel describing a state of a subject, according to aspects of the present disclosure.

FIG. 28 describes an example of data collected by force sensors, according to aspects of the present disclosure.

FIG. 29 shows a “summary” tab of a front panel, according to aspects of the present disclosure.

FIG. 30 shows a “Eyes Open” tab of the front panel, according to aspects of the present disclosure.

FIG. 31 shows a “Eyes Closed” tab of the front panel, according to aspects of the present disclosure.

FIG. 32 shows a “GRF_EO” tab of the front panel, according to aspects of the present disclosure.

FIG. 33 shows a “GRF_EC” tab of the front panel, according to aspects of the present disclosure.

FIG. 34A shows a loadcell sensor verification test for input resistance, according to aspects of the present disclosure.

FIG. 34B shows a loadcell sensor verification test for output resistance, according to aspects of the present disclosure.

FIG. 34C shows a loadcell sensor verification voltage test, according to aspects of the present disclosure.

FIG. 35 shows a mass being applied on a force platform, according to aspects of the present disclosure.

FIG. 36 shows an example of a lab device test, according to aspects of the present disclosure.

FIG. 37 shows an example of a force measurement platform test, according to aspects of the present disclosure.

FIG. 38 shows an example of the data of various subjects, according to aspects of the present disclosure.

FIG. 39A-FIG. 39C(b) show test results for a subject, according to aspects of the present disclosure.

FIG. 40A-FIG. 40C(b) show test results for another subject, according to aspects of the present disclosure.

FIG. 41A-FIG. 41C(b) show test results for yet another subject, according to aspects of the present disclosure.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of the present disclosure are directed to a force measurement platform for determining and monitoring postural stability of patients.

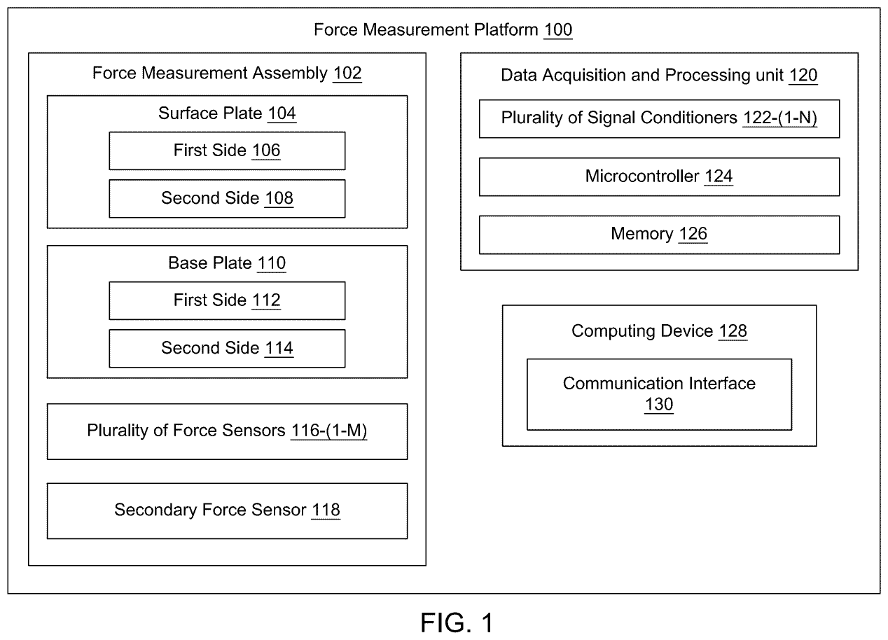

FIG. 1 depicts a block diagram of a force measurement platform 100, according to aspects of the present disclosure.

The force measurement platform 100 may be a platform configured to determine and monitor postural stability of patients. In an aspect, the force measurement platform 100 may monitor postures of patients during a static condition in order to quantify balance asymmetries. The force measurement platform 100 may be configured to measure ground reaction force (GRF) and center of pressure (COP). GRF represents the sum of all forces acting between a physical object (for example, a body) and its supporting surface. COP is defined as the point application of the GRF. The data obtained through measurement of the GRF and the COP may be analyzed to visualize COP sway and calculate its total path and velocity to provide further information about postural stability that may be used to determine any postural abnormality. The force measurement platform 100 may have a load capacity of up to 10 kilonewtons (kN). Also, the force measurement platform 100 meets the ANSI B157.1 standards and the ASTM F3109-16 standard

The force measurement platform 100 may include a force measurement assembly 102. The force measurement assembly 102 may include a surface plate 104. The surface plate 104 may include a first side 106 and a second side 108 opposite to the first side 106. The force measurement assembly 102 also includes a base plate 110. The base plate 110 includes a first side 112 and a second side 114 opposite to the first side 112. The second side 108 of the surface plate 104 may be above and opposite to the first side 112 of the base plate 110. In examples, each of the surface plate 104 and the base plate 110 may be of rectangular shape, square shape, or any other suitable shape. The design of surface plate 104 and the base plate 110 may be modeled using AutoCAD© software. Each of the surface plate 104 and the base plate 110 may be of a dimension of 500×500×8 mm. The surface plate 104 and the base plate 110 may be configured to measure the changes in voltage which is proportional to the force applied.

The surface plate 104 and the base plate 110 may be made of an aluminum alloy that is composed of, in % by mass, less than or equal to 0.4% of iron, less than or equal to 0.25% of silicon, and the balance (or remaining) being aluminum and unavoidable impurities. The surface plate 104 may be made of stainless steel, brass, heavy-duty polycarbonate plastic, and ultra-high molecular weight polyethylene plastic. The base plate 110 may be made of stainless steel, brass, heavy-duty polycarbonate plastic, and ultra-high molecular weight polyethylene plastic.

The force measurement platform 100 may further include a plurality of force sensors 116-(1-M) (interchangeably referred to as sensors 116-(1-M)). The plurality of force sensors 116-(1-M) may be mutually diagonally arranged in four quadrants of the force measurement assembly 102 with each quadrant containing at least one force sensor. An optional secondary force sensor 118 may be located in a geometric center of the force measurement assembly 102. In examples, the surface plate 104 may be supported on the base plate 110 via the plurality of force sensors 116-(1-M) and the optional secondary force sensor 118. The orientation of the plurality of force sensors 116-(1-M) and the secondary force sensor 118 helps in translating the magnitude and direction of forces which is GRF into electrical signals.

In an aspect, the plurality of force sensors 116-(1-M) and the optional secondary force sensor 118 may be a part of the force measurement assembly 102 (i.e., the force measurement assembly 102 may include the surface plate 104, the base plate 110, the plurality of force sensors 116-(1-M), and the secondary force sensor 118). In some aspects, the plurality of force sensors 116-(1-M) and the optional secondary force sensor 118 may not be a part of the force measurement assembly 102 (i.e., the force measurement assembly 102 may include only the surface plate 104 and the base plate 110). For the purposes of the present disclosure, the force measurement assembly 102 may be considered to be comprising the surface plate 104, the base plate 110, and the plurality of force sensors 116-(1-M).

Each of the plurality of force sensors 116-(1-M) may include a beam portion having a first end and a second end. The first end of the beam portion may be connected to the second side 108 of the surface plate 104 around at least one first pivot axis that is perpendicular to the surface plate 104, and may be configured to receive at least a portion of a load that is applied to the surface plate 104. The second end of the beam portion may be mounted to the first side 112 of the base plate 110 around at least one second pivot axis that is perpendicular to the base plate 110. The at least one first pivot axis may be parallel to the at least one second pivot axis.

For ease of explanation and understanding, description provided below may be with reference to four force sensors, however, the description is equally applicable to more than or less than four force sensors.

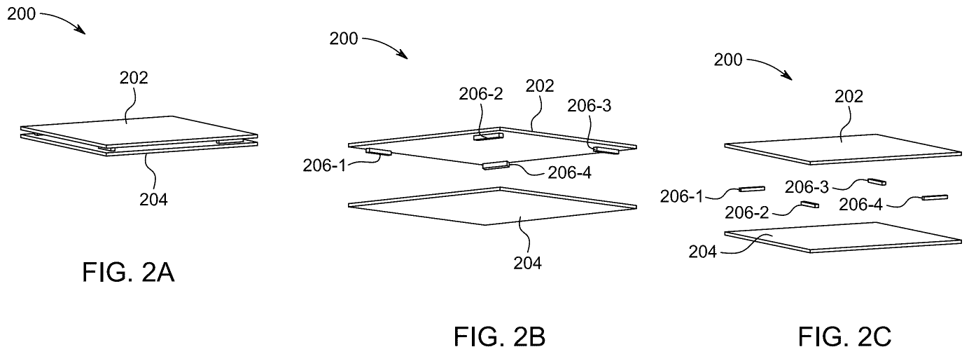

FIG. 2A-FIG. 2C illustrate isometric views of a force measurement assembly 200, according to aspects of the present disclosure. The force measurement assembly 200 may be an example of the force measurement assembly 102.

As shown in FIG. 2A-FIG. 2C, the force measurement assembly 200 may include a surface plate 202, a base plate 204, and four force sensors including a first force sensor 206-1, a second force sensor 206-2, a third force sensor 206-3, and a fourth force sensor 206-4. The surface plate 202 may be an example of the surface plate 104. The base plate 204 may be an example of the base plate 110. The force sensors 206-(1-4) may be examples of the force sensors 116-(1-4). The surface plate 202 includes a first side and a second side. The base plate 204 includes a first side and a second side. In the example shown in FIG. 2A, the surface plate 202 is placed above the base plate 204, where the second side of the surface plate 202 is above and opposite to the first side of the base plate 204.

In the example shown in FIG. 2B, the first force sensor 206-1, the second force sensor 206-2, the third force sensor 206-3, and the fourth force sensor 206-4 are attached to the second side of the surface plate 202 in four quadrants of the surface plate 202 with each quadrant containing one force sensor; however, this should not be construed as a limitation. In some aspects, the first force sensor 206-1, the second force sensor 206-2, the third force sensor 206-3, and the fourth force sensor 206-4 may be attached to the first side of the base plate 204. FIG. 2C shows the surface plate 202, base plate 204, and the force sensors 206-(1-4) separately.

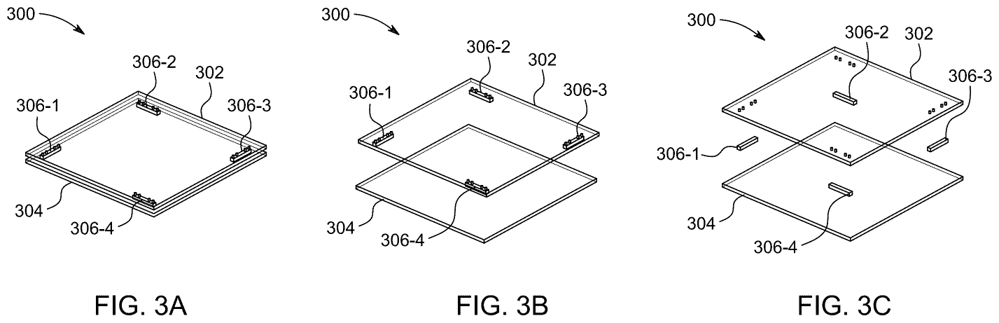

FIG. 3A-FIG. 3C illustrate wireframe isometric views of a force measurement assembly 300, according to aspects of the present disclosure. The force measurement assembly 300 may be an example of the force measurement assembly 102.

As shown in FIG. 3A-FIG. 3C, the force measurement assembly 300 may include a surface plate 302, a base plate 304, and four force sensors including a first force sensor 306-1, a second force sensor 306-2, a third force sensor 306-3, and a fourth force sensor 306-4. The surface plate 302 may be an example of the surface plate 104. The base plate 304 may be an example of the base plate 110. The force sensors 306-(1-4) may be examples of the force sensors 116-(1-4). The surface plate 302 includes a first side and a second side. The base plate 304 includes a first side and a second side. In the example shown in FIG. 3A, the surface plate 302 is placed above the base plate 304, where the second side of the surface plate 302 is above and opposite to the first side of the base plate 304.

In the example shown in FIG. 3B, the first force sensor 306-1, the second force sensor 306-2, the third force sensor 306-3, and the fourth force sensor 306-4 are attached to the second side of the surface plate 302 in four quadrants of the surface plate 302 with each quadrant containing one force sensor. FIG. 3C shows the surface plate 302, base plate 304, and the force sensors 306-(1-4) separately.

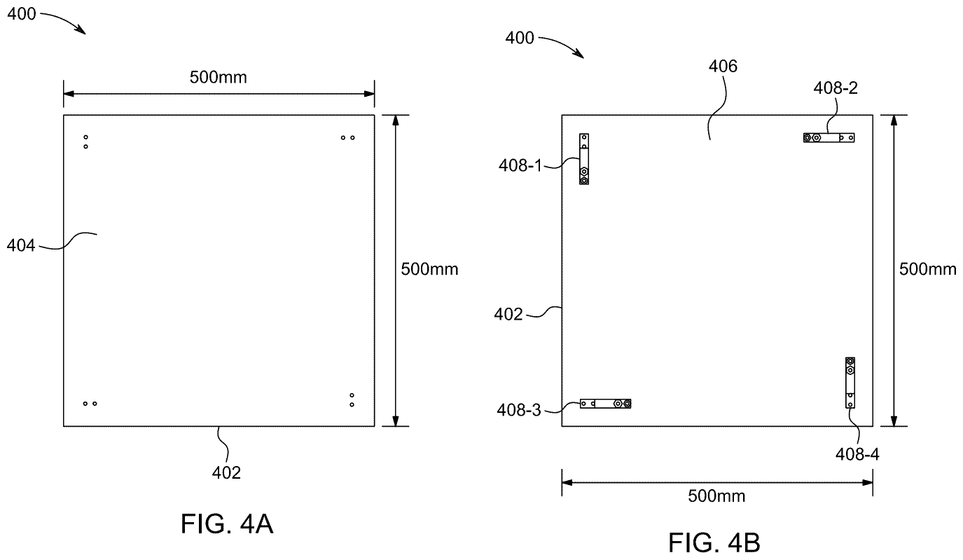

FIG. 4A shows a top view of a force measurement assembly 400, according to aspects of the present disclosure. In FIG. 4A, a surface plate 402 having a first side 404 is shown. The surface plate 402 has a size of about 500×500 mm. The force measurement assembly 400 may be an example of the force measurement assembly 102. The surface plate 302 may be an example of the surface plate 104.

FIG. 4B shows the force measurement assembly 400 comprising force sensors, according to aspects of the present disclosure. In FIG. 4B, the surface plate 402 having a second side 406 is shown. As shown in FIG. 4B, four sensors including a first force sensor 408-1, a second force sensor 408-2, a third force sensor 408-3, and a fourth force sensor 408-4 are attached to corners of the second side 406 of the surface plate 402, where one force sensor is attached to each corner. The force sensors 408-(1-4) may be examples of force sensors 116-(1-N).

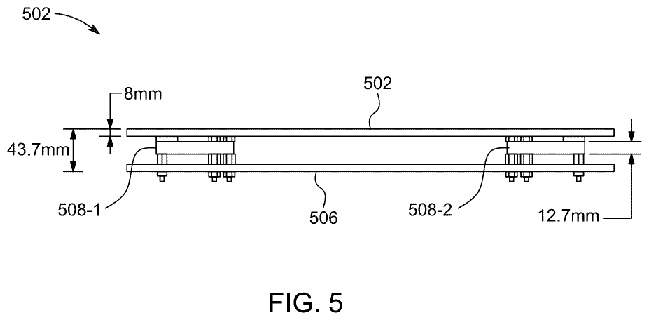

FIG. 5 shows a side view of a force measurement assembly 502, according to aspects of the present disclosure. The force measurement assembly 502 may be an example of the force measurement assembly 102. The force measurement assembly 502 includes a surface plate 504 and a base plate 506. The surface plate 504 may be an example of the surface plate 104. The base plate 506 may be an example of the base plate 110.

As shown in FIG. 5, thickness of each of the surface plate 504 and the base plate 506 is about 5 to 20 mm, preferably about 8 mm. Further, in the side view of the force measurement assembly 502, two force sensors including a first force sensor 508-1 and a second force sensor 508-2 are visible. Each of the first force sensor 508-1 and the second force sensor 508-2 may have a thickness of about 6 to 20 mm, preferably about 12.7 mm. In the side view of the force measurement assembly 502, only two force sensors (i.e., the first force sensor 508-1 and the second force sensor 508-2) are visible, though the force measurement assembly 502 may include more than two force sensors. A total thickness of the force measurement assembly 502 may be about 30 to 50 mm, preferably about 43.7 mm. Other ranges are also possible.

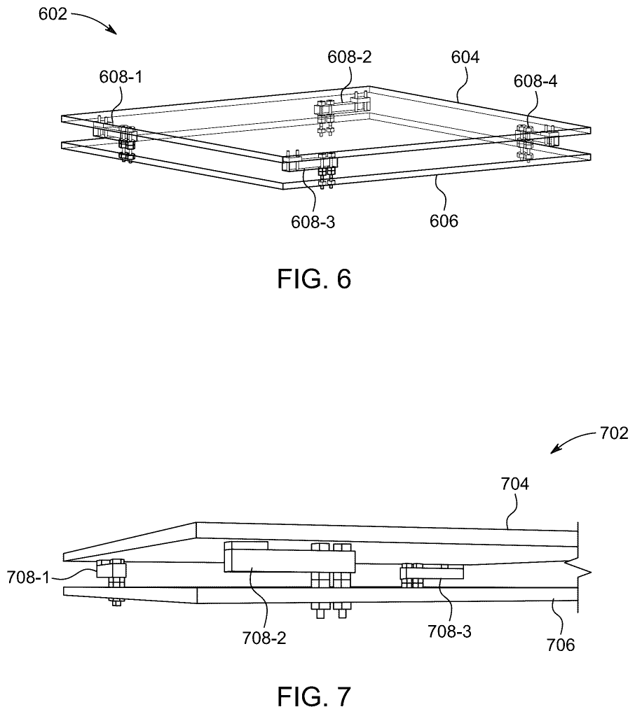

FIG. 6 shows an isometric view of a force measurement assembly 602, according to aspects of the present disclosure. The force measurement assembly 602 may be an example of the force measurement assembly 102. The force measurement assembly 602 includes a surface plate 604, a base plate 606, and four force sensors include a first force sensor 608-1, a second force sensor 608-2, a third force sensor 608-3, and a fourth force sensor 608-4. The surface plate 604 may be an example of the surface plate 104. The base plate 606 may be an example of the base plate 110. The force sensors 608-(1-4) may be examples of force sensors 116-(1-4).

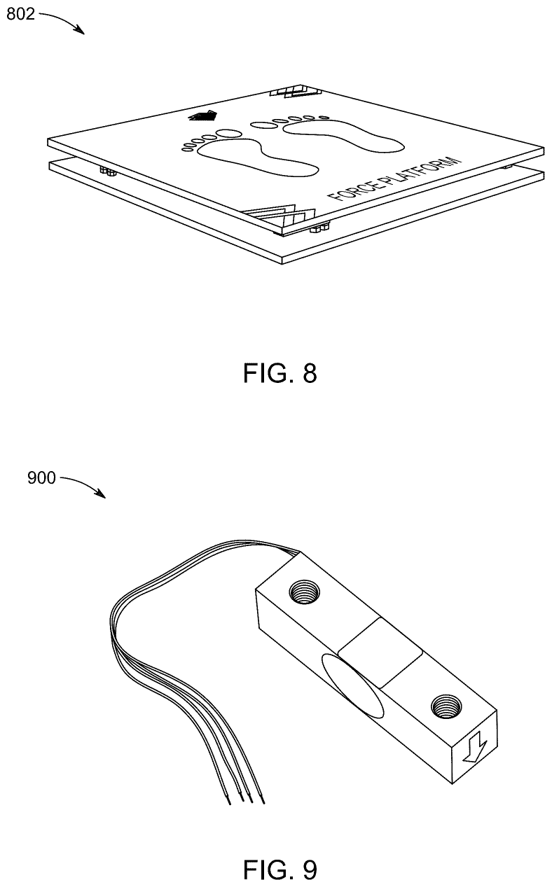

FIG. 7 shows a close-up view of a force measurement assembly 702, according to aspects of the present disclosure. The force measurement assembly 702 may be an example of the force measurement assembly 102. The force measurement assembly 702 includes a surface plate 704 and a base plate 706. Further, in the close-up view of the force measurement assembly 702, three force sensors including a first force sensor 708-1, a second force sensor 708-2, and a third force sensor 708-3, are visible. The surface plate 704 may be an example of the surface plate 104. The base plate 706 may be an example of the base plate 110. The force sensors 708-(1-3) may be examples of force sensors 116-(1-3).

FIG. 8 shows an isometric view of a force measurement assembly 802, according to aspects of the present disclosure. The force measurement assembly 802 may be an example of the force measurement assembly 102.

Referring back to FIG. 1, each of the plurality of force sensors 116-(1-M) may include a plurality of deformation sensing elements disposed on the beam portion of the force sensor along a longitudinal axis between the first end and the second end of the beam portion. The plurality of deformation sensing elements may covert an input mechanical load to an electrical signal representative of the load that is applied to the surface plate 104. The force sensor may be at least one selected from the group consisting of a capacitive displacement sensor, an inductive force sensor, a strain gauge, a piezoelectric force sensor, a force sensing resistor, a piezoresistive force sensor, a thin film force sensor, and a quantum tunnelling composite-based force sensor.

The optional secondary force sensor 118 may include a beam portion having a first end and a second end. The first end of the beam portion may be connected to the second side 108 of the surface plate 104 around at least one first pivot axis that is perpendicular to the surface plate 104, and is configured to receive at least a portion of a load that is applied to the surface plate 104. The second end of the beam portion may be mounted to the first side 112 of the base plate 110 around at least one second pivot axis that is perpendicular to the base plate 110. The at least one first pivot axis is parallel to the at least one second pivot axis. Further, the optional secondary force sensor 118 may include a plurality of deformation sensing elements disposed on the beam portion of the optional secondary force sensor 118 along a longitudinal axis between the first end and the second end of the beam portion. The plurality of deformation sensing elements may convert an input mechanical load to an electrical signal representative of the load that is applied to the surface plate 104. The optional secondary force sensor may be at least one selected from the group consisting of a capacitive displacement sensor, an inductive force sensor, a strain gauge, a piezoelectric force sensor, a force sensing resistor, a piezoresistive force sensor, a thin film force sensor, and a quantum tunneling composite-based force sensor.

An example of strain gauge sensor includes loadcell TAL220 sensor (manufactured by HT sensor technology co. ltd., Nr. 2, JinyeRoad, Xi'an-710077 P.R. CHINA). The loadcell TAL220 sensor is a 200 kg sensor that can measure loads ranging from 0 to 200 kg. The main characteristics of the loadcell TAL220 sensor are summarized in Table 1 provided below.

TABLE 1
Characteristic of loadcell TAL220 sensor

| Capacity | Material | Type | Application |
|---|---|---|---|
| 3-200 kg | Aluminum-alloy or | Parallel | Electronic platform scale. |
| | alloy steel | beam | Electronic weighing devices. |
| | | | Electronic balance. |

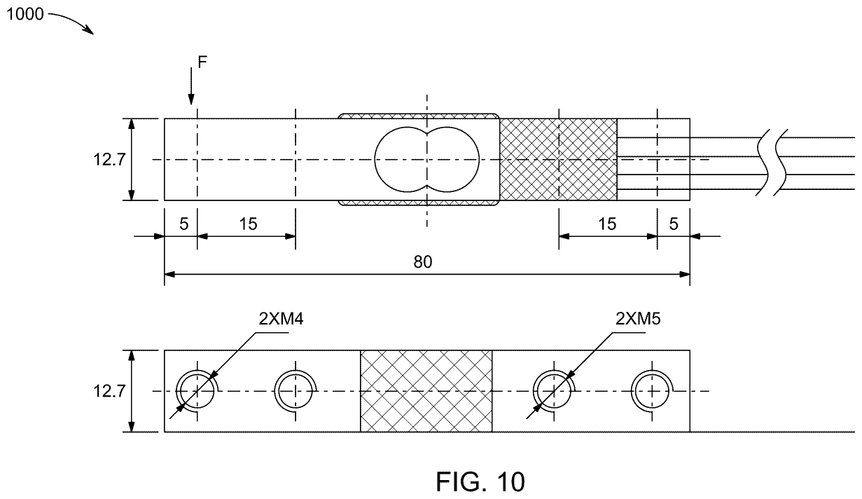

FIG. 9 describes a loadcell TAL220 sensor 900, according to aspects of the present disclosure, and FIG. 10 describes an example 1000 of load cell dimensions, according to aspects of the present disclosure.

Referring back to FIG. 1, the force measurement platform 100 may include a data acquisition and processing unit 120. The data acquisition and processing unit 120 may be configured to receive one or more signals that are representative of a load being applied to a surface of the force measurement assembly 102 and to record a location of the applied load on the surface of the force measurement assembly 102 by computing a center of pressure (COP) for the applied load as determined by ASTM F3109-16 standard. The data acquisition and processing unit 120 may be further configured to monitor the signals of the applied load over time to visualize a sway or a change in the location of the load being applied on the surface of the force measurement assembly 102 by computing the COP.

The data acquisition and processing unit 120 may include a plurality of signal conditioners 122-(1-N) and a microcontroller 124. The microcontroller 124 may include a memory 126 for saving or installing an application program, or a software program. The application program or the software program may be downloaded from the Internet or a cloud server for interpreting and executing digital signals. In examples, each of the plurality of signal conditioners 122-(1-N) may be operatively connected to each of the plurality of force sensors 116-(1-M) and the optional secondary force sensor 118 of the force measurement assembly 102. The plurality of signal conditioners 122-(1-N) may be operatively connected to the microcontroller 124. In examples, the plurality of signal conditioners 122-(1-N) comprises at least one amplifier, at least one high-pass filter, and at least one low-pass filter.

In examples, a signal conditioner may be an HX711 load cell amplifier (manufactured by AVIA Semiconductor Ltd., Chengyi Rd, Xiamen Software Park Phase 3, Xiamen, P. R. China). The HX711 load cell amplifier may be configured to magnify the signals produced by the force sensors 116-(1-M) and convert them to a digital signal.

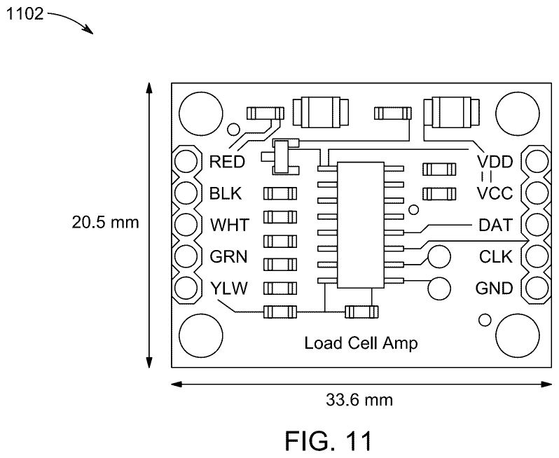

FIG. 11 depicts a HX711 load cell amplifier 1102, according to aspects of the present disclosure. The HX711 load cell amplifier 1102 is a 24-bit Analog-to-Digital converter (ADC) that may be used to get measurable data by reading a force sensor, for example, load cell TAL220 sensor. In an example, size of the HX711 load cell amplifier 1102 may be 20.5×33.6 mm. The HX711 load cell amplifier 1102 may be connected to a microcontroller such as Arduino from a right side to read the data that comes from the force sensor from a left side.

Referring back to FIG. 1, the force measurement platform 100 also includes a computing device 128. The computing device 128 may include a personal computer (PC), a server, a mobile computing device, and a computing circuit. The computing device 128 may include a communication interface 130 (interchangeably referred to as communication interface 130) coupled with the data acquisition and processing unit 120. Although, the computing device 128 is described to be a part of the force measurement platform 100, the computing device 128 may be implemented external to the force measurement platform 100. In examples, the force measurement platform 100 may removably retain the computing device 128. The computing device 128 may present a data visualization configuration interface according to a load applied to the force measurement platform 100. The computing device 128 may be connected to the data acquisition and processing unit 120 via a USB cable.

In an example, the data acquisition and processing unit 120 may be Arduino. The Arduino is an open-source electronic platform that may be used to acquire a digital signal produced by force sensors, such as load cells after being amplified. In examples, analog signals that represent forces measured by each force sensor may be converted to digital signals through an amplifier kit (HX711). Then, the data acquisition and processing unit may acquire the data 120. The data acquisition and processing unit 120 may be connected to the computing device 128 with LabVIEW software program (explained later in the description) through a USB cable to visualize the data. The LabVIEW software program is built by NI Inc, 11500 N Mopac Expwy, Austin, TX 78759-3504, USA.

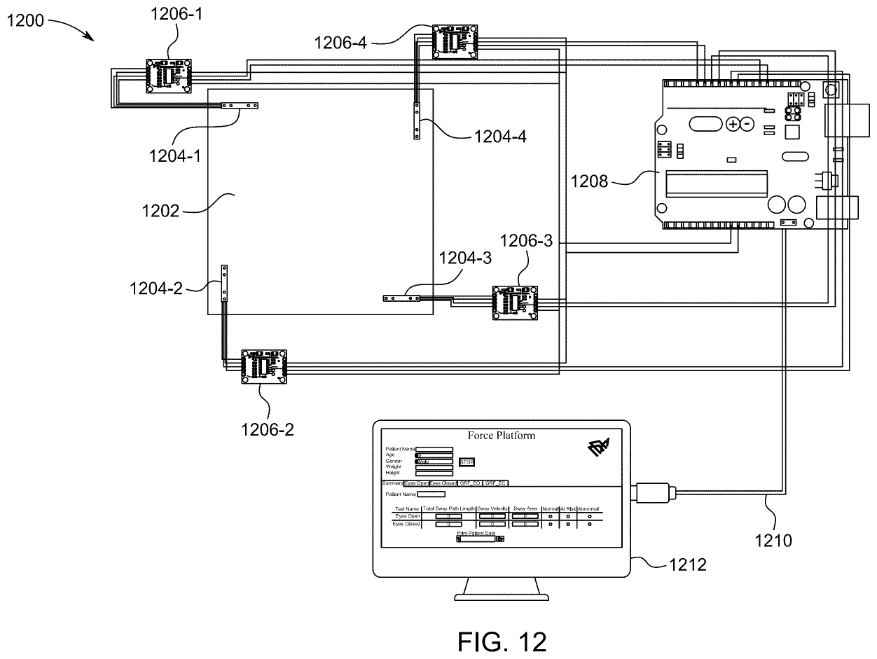

FIG. 12 depicts electrical connections of a force measurement platform 1200, according to aspects of the present disclosure. The force measurement platform 1200 may be an example of the force measurement platform 100. The force measurement platform 1200 includes force plate 1202. The force plate 1202 may be a surface plate (for example, surface plate 104) or a base plate (for example, a base plate 110). The force measurement platform 1200 includes four force sensors including a first force sensor 1204-1, a second force sensor 1204-2, a third force sensor 1204-3, and a fourth force sensor 1204-4, where the four force sensors 1204-(1-4) are attached to the force plate 1202. The force sensors 1204-(1-4) may be examples of the force sensors 116-(1-4). In example, the force sensors 1204-(1-4) may be TAL220 loadcells sensors. Each force sensor is connected to a signal conditioner. In an example, the first force sensor 1204-1 is connected to a first signal conditioner 1206-1, the second force sensor 1204-2 is connected to a second signal conditioner 1206-2, the third force sensor 1204-3 is connected to a third signal conditioner 1206-3, and the fourth force sensor 1204-4 is connected to a fourth signal conditioner 1206-4. In an example, signal conditioners 1206-(1-4) may be loadcell amplifier HX711. The force measurement platform 1200 also include a microcontroller 1208 and a computing device 1212. The microcontroller 1208 may be connected to the computing device 1212 via a USB cable 1210. In an example, the microcontroller 1208 may be the Arduino microcontroller (by Arduino LLC, 265 Franklin street suite 1702, Boston, MA 02110 USA), and the computing device 1212 may be a PC.

According to an aspect, prior to determining and monitoring postural stability of a patient, the force measurement platform 100 may be calibrated. A load may be applied to a surface of the force measurement assembly 102 via at least one portion of a body of the patient. The force measurement platform 100 may be run for 1 to 120 seconds, preferably 5 to 60 seconds, or even more preferably 10 to 30 seconds to calculate a COP and record a sway. The results of COP and sway may be compared against a reference database to determine and monitor postural stability. Other ranges are also possible.

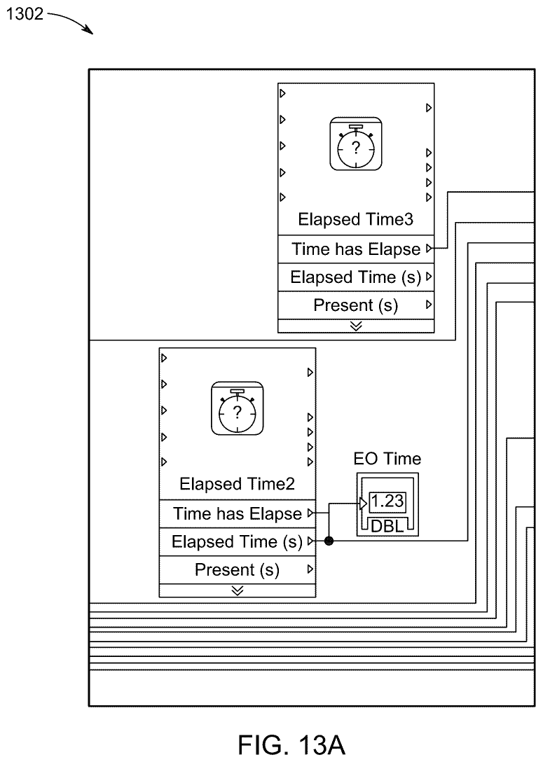

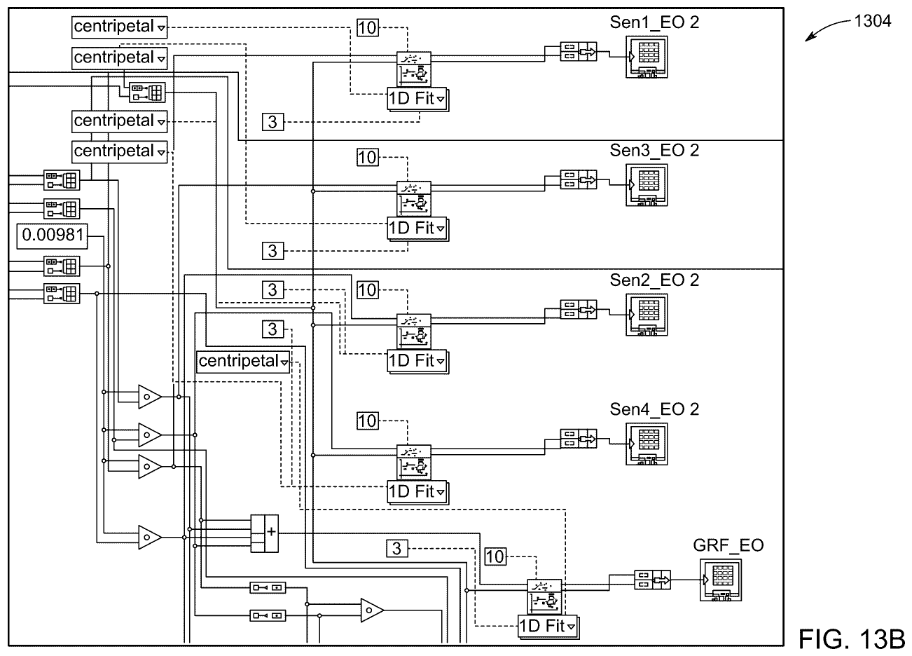

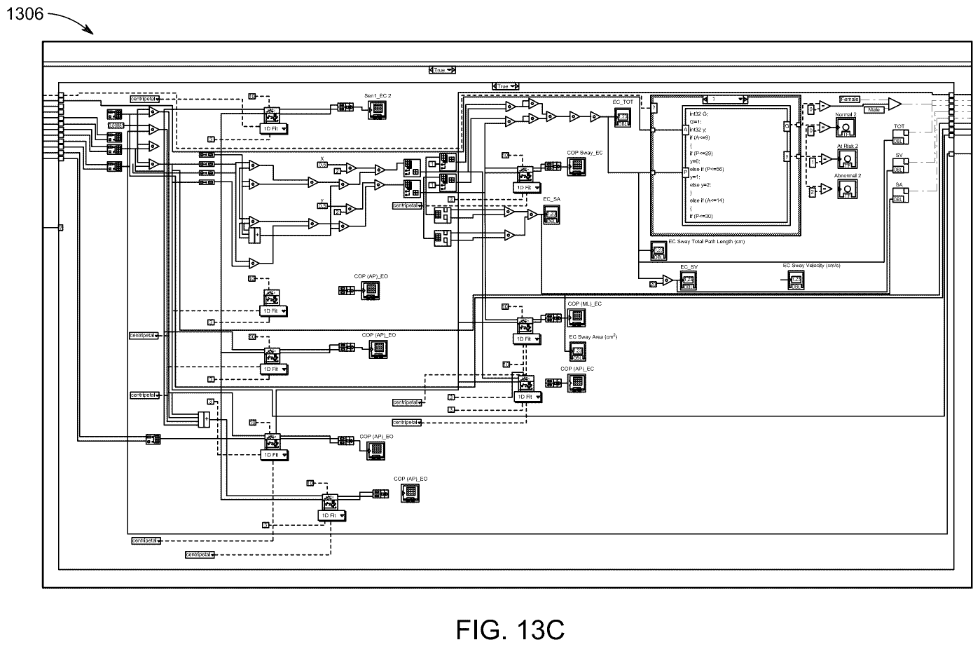

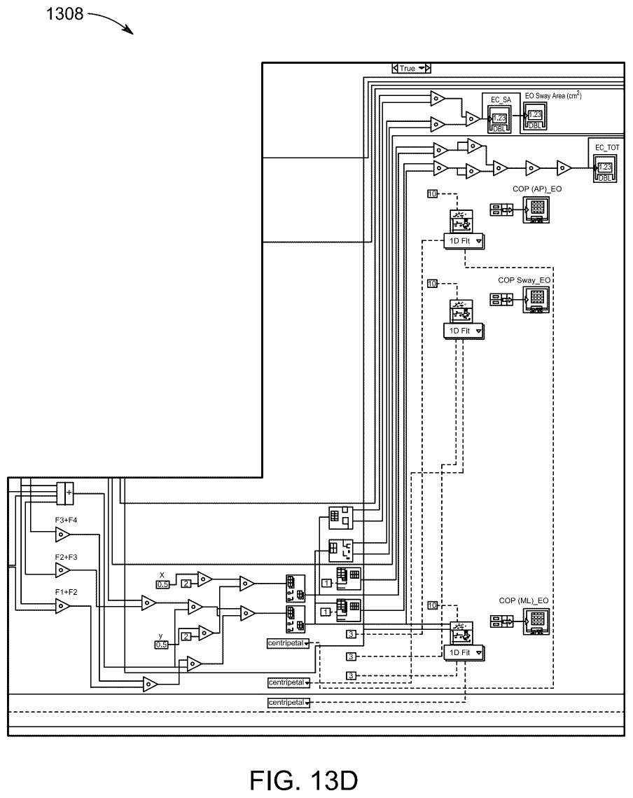

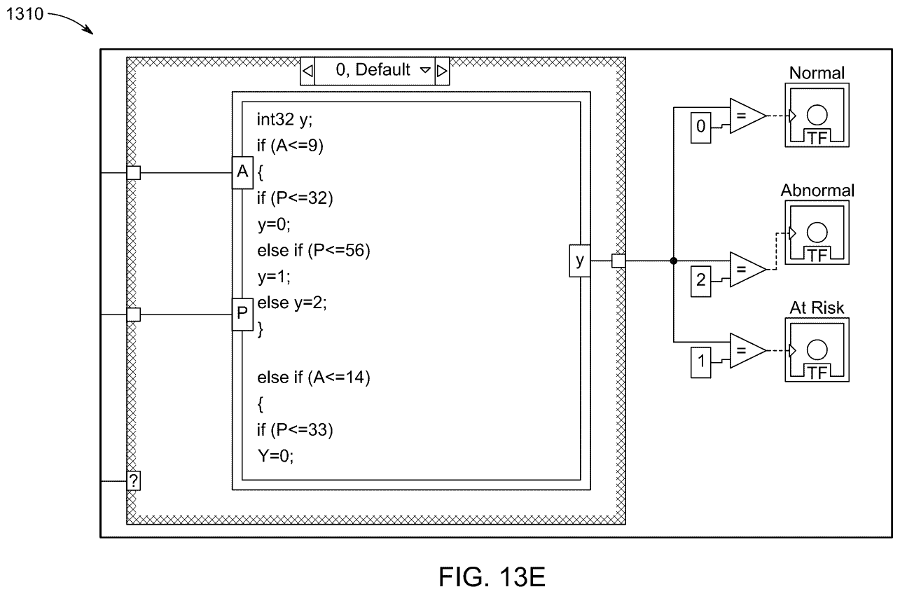

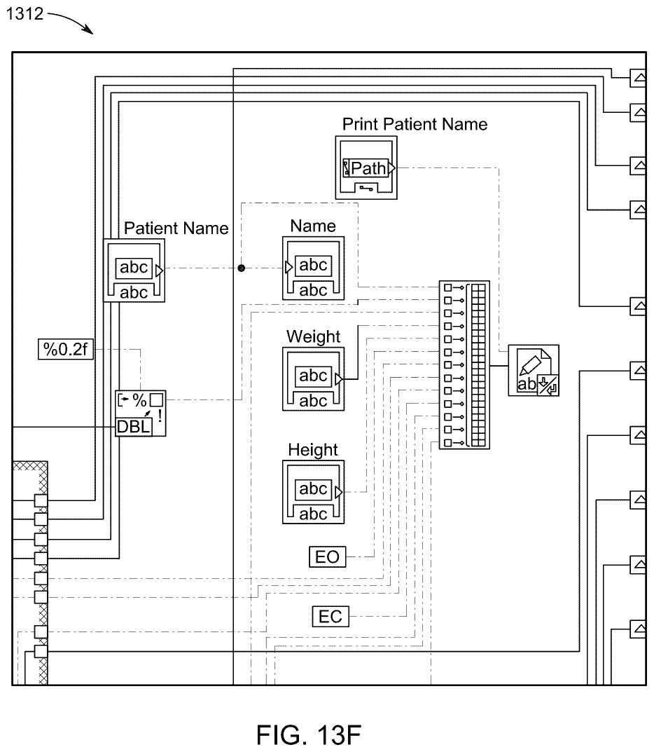

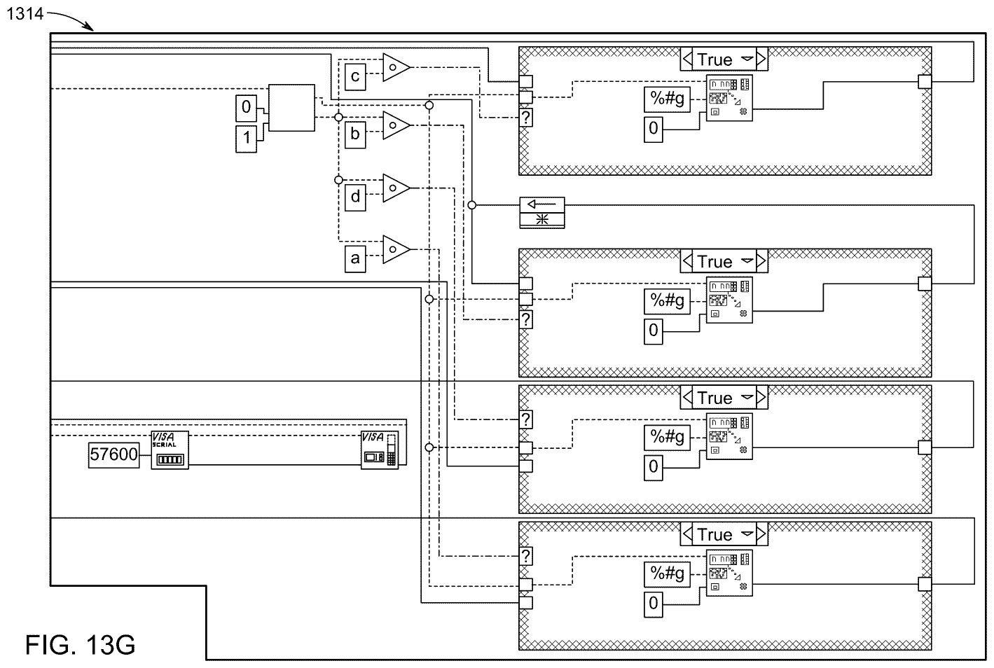

In examples, a LabVIEW software program (interchangeably referred to as a program) includes a block diagram and a front panel. The block diagram is a graphical representation of the data flow within the program. FIGS. 13A-13G show different sections of a block diagram of a LabVIEW software program for monitoring the GRF, calculating the COP, and COP sway, according to aspects of the present disclosure. In particular, FIG. 13A shows test timing section 1302 of the block diagram. FIG. 13B shows input signals in section 1304 of the block diagram. FIG. 13C shows another section 1306 of the block diagram. FIG. 13D shows COP calculation section 1308 of the block diagram. FIG. 13E shows database comparison section 1310 of the block diagram. FIG. 13F shows the result print section 1312 of the block diagram. FIG. 13G shows VISA connection section 1314 of the block diagram.

An example of the code is shown below. HX711_ADC is the Arduino library used along with the HX711 amplifier for load cells. The code is used to operate the four load cells (sensors) together.

```
#include <HX711_ADC.h>
#if defined(ESP8266)|| defined(ESP32) || defined(AVR)
#include <EEPROM.h>
#endif
//Assigning pins to each amplifiers' output :
const int data1 = 12; // Sensor_1 data
const int clock1 = 13; // Sensor_1 clock
const int data2 = 8; // Sensor_2 data
const int clock2 = 9; // Sensor_2 clock
const int data3 = 6; // Sensor_3 data
const int clock3 = 7; // Sensor_3 clock
const int data4 = 4; // Sensor_4 data
const int clock4 = 5; // Sensor_4 clock
//HX711 identifies each loadcell:
HX711_ADC Sensor_1(data1, clock1);
HX711_ADC Sensor_2(data2, clock2);
HX711_ADC Sensor_3(data3, clock3);
HX711_ADC Sensor_4(data4, clock4);
unsigned long timing = 0;
void setup( ) {
  Serial.begin(57600); delay(10);
  Serial.println( );
  Serial.println(“Starting...”);
  Sensor_1.begin( );
  Sensor_2.begin( );
  Sensor_3.begin( );
  Sensor_4.begin( );
  unsigned long time_stable = 2000;
  boolean for_tare = true;
  Sensor_1.start(time_stable, for_tare);
  Sensor_2.start(time_stable, for_tare);
  Sensor_3.start(time_stable, for_tare);
  Sensor_4.start(time_stable, for_tare);
  if (Sensor_1.getTareTimeoutFlag( ) || Sensor_1.getSignalTimeoutFlag( )) {
    Serial.println(“Timeout! check HX711 to Arduino wiring and pin configuration”);
    while (1);
  }
  else {
    Sensor_1.setCalFactor(9.89);
    Serial.println(“Starting 1 Done”);
  }
  if (Sensor_2.getTareTimeoutFlag( ) || Sensor_2.getSignalTimeoutFlag( )) {
    Serial.println(“Timeout! check HX711 to Arduino wiring and pin configuration”);
    while (1);
  }
  else {
    Sensor_2.setCalFactor(7.72);
    Serial.println(“Starting 2 Done”);
  }
    if (Sensor_3.getTareTimeoutFlag( ) || Sensor_3.getSignalTimeoutFlag( )) {
      Serial.println(“Timeout! check HX711 to Arduino wiring and pin configuration”);
      while (1);
  }
  else {
      Sensor_3.setCalFactor(8.24);
      Serial.println(“Starting 3 Done”);
    }
    if (Sensor_4.getTareTimeoutFlag( ) || Sensor_4.getSignalTimeoutFlag( )) {
      Serial.println(“Timeout! check HX711 to Arduino wiring and pin configuration”);
      while (1);
  }
    else {
      Sensor_4.setCalFactor(7.55);
      Serial.println(“Starting 4 Done”);
  }
    while (!Sensor_1.update( ));
    while (!Sensor_2.update( ));
    while (!Sensor_3.update( ));
  while (!Sensor_4.update( ));
  }
  void loop( ) {
    const int activitySerial = 0; //to set the activity of the serial print
    static boolean searchNewdata = 0;
    // Searching for new data:
    if (Sensor_1.update( )) searchNewdata = true;
    if (Sensor_2.update( )) searchNewdata = true;
    if (Sensor_3.update( )) searchNewdata = true;
    if (Sensor_4.update( )) searchNewdata = true;
    // obtain smoothed data from the dataset:
  if (searchNewdata) {
    if (millis( ) > timing + activitySerial) {
        float D_1 = Sensor_1.getData( );
        Serial.print(D_1);
      float D_2 = Sensor_2.getData( );
      Serial.print(‘\t’);
      Serial.print(D_2);
      float D_3 = Sensor_3.getData( );
      Serial.print(‘\t’);
      Serial.print(D_3);
      float D_4 = Sensor_4.getData( );
      Serial.print(‘\t’);
      Serial.println(D_4);
      searchNewdata = 0;
      timing = millis( );
    }
  }
```

According to the LabVIEW software program, the data acquired from the Arduino Uno using VISA, which represents the forces may be plotted to allow an end-user to visualize them. Then, the forces may be used to calculate and plot anterior-posterior component of COP (denoted as “COP_{AP}”) and medial-lateral component of COP (denoted as “COP_{ML}”) which may be used to visualize the movement of the COP as COP sway. Finally, COP sway total path length (denoted as “S_{TOT}”), COP velocity (denoted as “V_{COP}”), and COP sway velocity (denoted as “A_{sway}”) are calculated using Equations (1), (2), (3), (4), and (5), respectively.

$${S}_{TOT} = {\sum }_{i = 1}^{N} \sqrt{{( {COP}_{{AP}_{( i + 1 )}} - {COP}_{{AP}_{( i )}} )}^{2} + {( {COP}_{{ML}_{( i + 1 )}} - {COP}_{{ML}_{( i )}} )}^{2}} \qquad ( 1 )$$

$${COP}_{AP} = \frac{x}{2} \times \frac{( {F}_{1} + {F}_{4} ) - ( {F}_{2} + {F}_{3} )}{{F}_{z}} where , \\ {COP}_{AP} = \frac{x}{2} \times \frac{( {F}_{1} + {F}_{4} ) - ( {F}_{2} + {F}_{3} )}{{F}_{z}} \qquad ( 2 )$$

$${COP}_{AP} = \frac{x}{2} \times \frac{( {F}_{1} + {F}_{4} ) - ( {F}_{2} + {F}_{3} )}{{F}_{z}} \qquad ( 3 )$$

where, COP_{AP}=anterior-posterior component of force plate, COP_{ML}=medial-lateral component of force plate, x=width of the functioning space of the plate, y=length of the functioning space of the plate, F_{z}=summation of all vertical reaction forces, and F₁ to F₄=forces measured by each sensor.

$${V}_{COP} = \frac{{S}_{TOT}}{{T}_{trial}} \qquad ( 4 )$$

$${A}_{sway} = ( {COP}_{AP ( m )} - {COP}_{AP ( m )} ) \times ( {COP}_{{ML}_{( m )}} - {COP}_{ML ( m )} ) \qquad ( 5 )$$

where N=the total number of data and Tₜᵣᵢₐₗ=the duration of the trial

In an example, Table 2 summarizes the main functions used in the LabVIEW software program.

TABLE 2
Main functions used in LabVIEW software program

| Function | Role |
|---|---|
| Index Array index | Returns the 2D array elements at |
| Array Max & Min | Obtains the maximum and minimum values of an array |
| Bundle | Combines individual elements or arrays of elements together |
| Select | Selects the ‘t’ when s is TRUE. Otherwise ‘f’ is selected |
| Elapsed Time | Indicates the time elapsed since the user pressed start |
| Numeric Functions | Performs the basic arithmetic operations including addition, |
| | subtraction, multiplications, division, square root and summation |
| Write to Text File | Writes the data connected and saves it as a text file |
| VISA Configure | Recognizes the serial port identified by the resource name |
| Serial Port | |
| VISA Set I/O | Assigns the I/O buffer size |
| Buffer Size | |
| Replace Substring | Replaces or deletes the substring at the specified offset |
| Scan Value | Converts the string into numerical data |

In examples, front panel (i.e, graphical user interface) includes different controls and indicators to allow the end-user to effectively interact with the program. The front panel may be an example of the data visualization configuration interface 130.

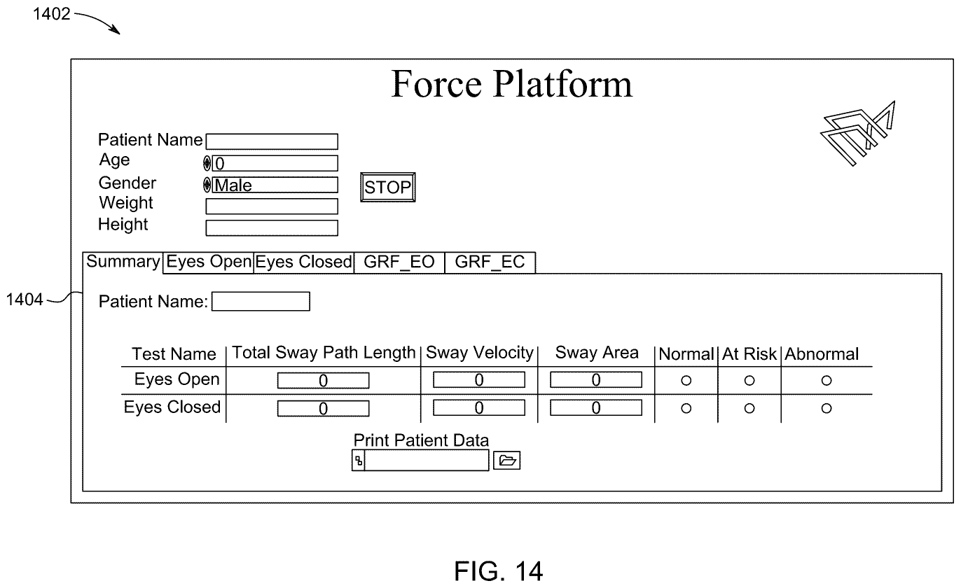

FIG. 14 shows a front panel 1402 of a LabVIEW software program, according to aspects of the present disclosure. The front panel 1402 includes an entry area to allow a physician to enter patient information. The patient information is to be filled in by the physician. As shown in FIG. 14, the front panel 1402 includes a “Summary” tab 1504. When the “Summary” tab is clicked, the patient measurement summary is shown. The patient measurement summary summarizes the results of the two tests performed by the patient i.e., the “Eyes Open” test and the “Eyes Closed” test, and allows the end-user to print all of these information in one text file.

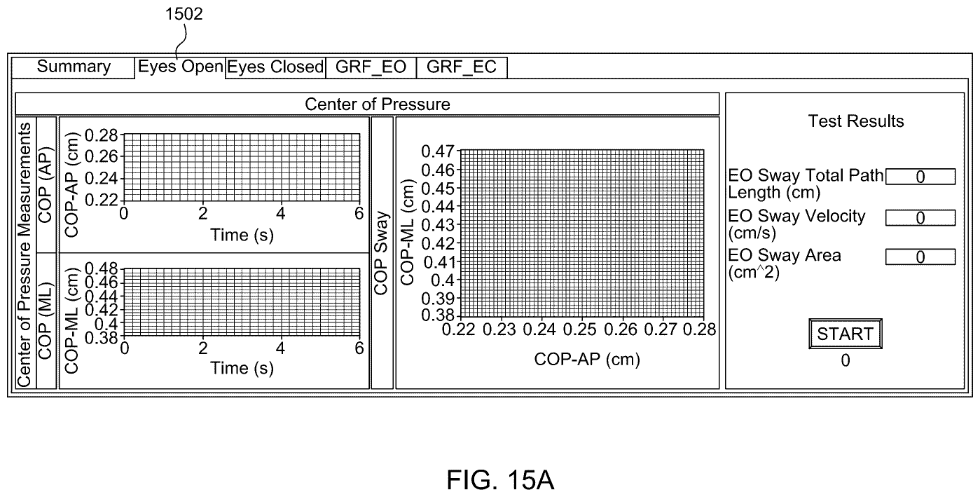

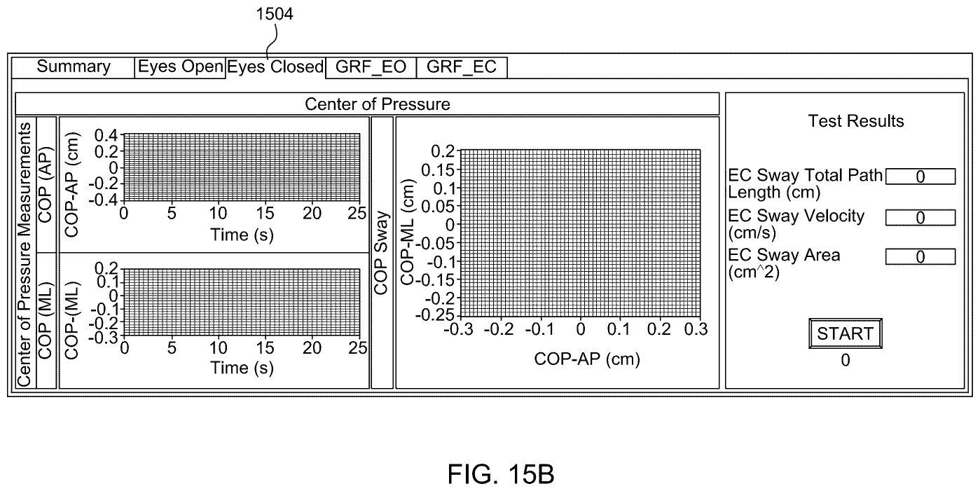

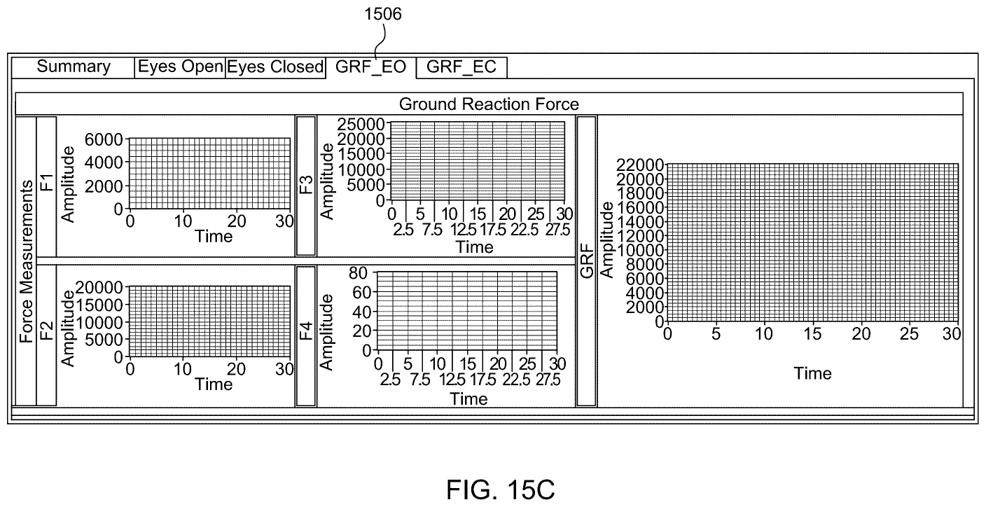

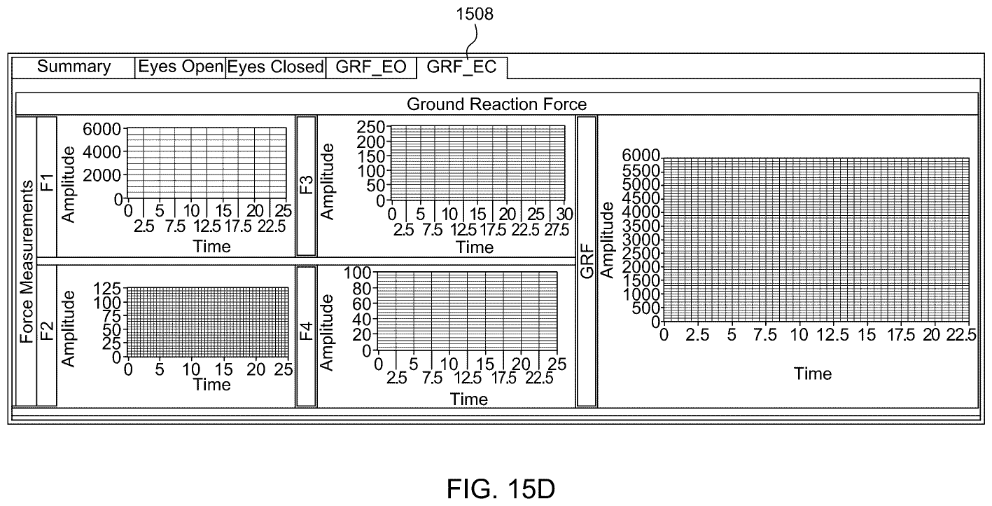

FIG. 15A-FIG. 15D show different tabs of the front panel 1402 of the LabVIEW software program, according to aspects of the present disclosure.

FIG. 15A shows a “Eyes Open” tab 1502. The “Eyes Open” tab 1502 specifies the COP sway details including S_{TOT}, V_{COP} and A_{sway} for “Eyes Open” test. FIG. 15B shows a “Eyes Closed” tab 1504. The “Eyes Closed” tab 1504 specifies the COP sway details including S_{TOT}, V_{cop} and A_{sway} for “Eyes Closed” test. FIG. 15C shows a “GRF_EO” tab 1506. The “GRF_EO” tab 1506 includes the plots of GRF components for “Eyes Open” test. FIG. 15D shows a “GRF_EC” tab 1508. The “GRF_EC” tab 1508 includes the plots of GRF components for “Eyes Closed” test.

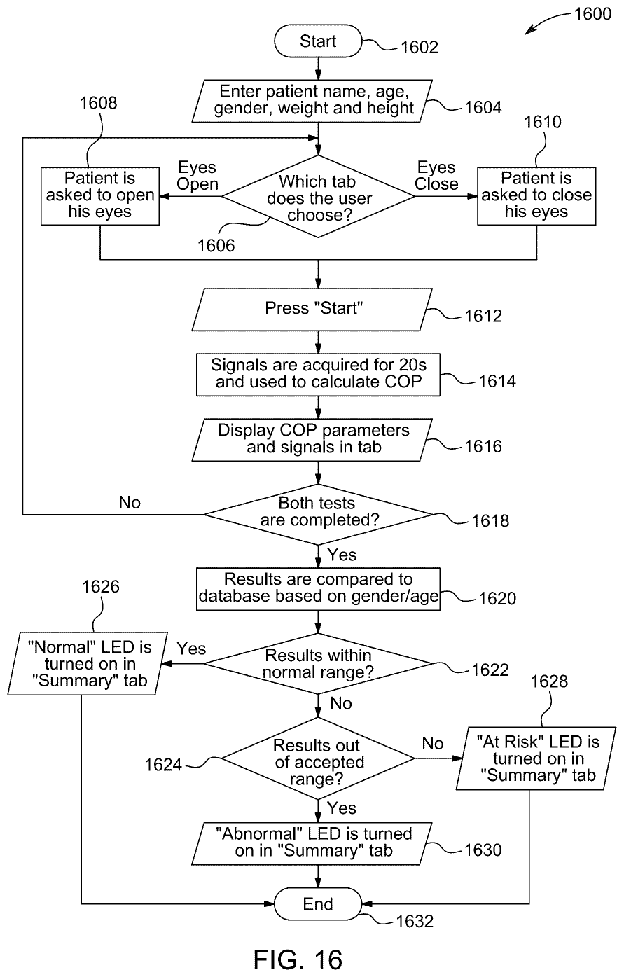

FIG. 16 illustrates a flowchart 1600 of the LabVIEW software program, according to aspects of the present disclosure.

At step 1602, the flowchart 1600 begins at step 1602. In an example, a patient may be asked to stand on a force measurement assembly of a force measurement platform(for example, the force measurement platform 100). At step 1604, patient's name, age, gender, weight, and height may be entered in a front panel of the LabVIEW software program. In an example, these details may be entered by a user (for example, a physician). At step 1606, it is determined which tab the user chooses (i.e., whether the user chooses an “Eyes Open” tab or an “Eyes Closed” tab of the front panel). If the user chooses, the “Eyes Open” tab, then the flowchart 1600 may proceed to step 1608. If the user chooses, the “Eyes Closed” tab, then the flowchart 1600 may proceed to step 1610.

At step 1608, the patient is asked to open his or her eyes. At step 1610, the patient is asked to close his or her eyes. At step 1612, the user is prompted to press a “start” icon on the front panel. At step 1614, signals are acquired for 20s and the signals are used to calculate COP. At step 1616, COP parameters and signals are displayed. At step 1618, it is determined if both tests (i.e., “Eyes Open” test and “Eyes Closed” test) are completed. If both tests are completed, then the flowchart 1600 proceeds to step 1620 (‘Yes’ Branch). If both tests are not completed, then the flowchart 1600 again starts from step 1606 (‘No’ Branch). At step 1620, the results of the test are compared to a database based on gender or age. At step 1622, it is determined if the results are within a normal range. If the results are not within the normal range, the flowchart 1600 proceeds to step 1624 (‘No’ Branch). If the result is within the normal range, the flowchart 1600 proceeds to step 1626 (‘Yes’ Branch). At step 1626, “Normal” LED is turned on in “Summary” tab of the front panel and the flowchart 1600 ends at step 1632. At step 1624, it is determined whether the results are out of accepted range. If it is determined that the results are not out of accepted range, then the flowchart 1600 proceeds to step 1628 (‘No’ Branch). If it is determined that the results are out of accepted range, then the flowchart 1600 proceeds to step 1630 (‘Yes’ Branch). At step 1628, “At Risk” LED is turned on in “Summary” tab and the flowchart 1600 ends at step 1632. At step 1630, “Abnormal” LED is turned on in “Summary” tab” and the flowchart 1600 ends at step 1632.

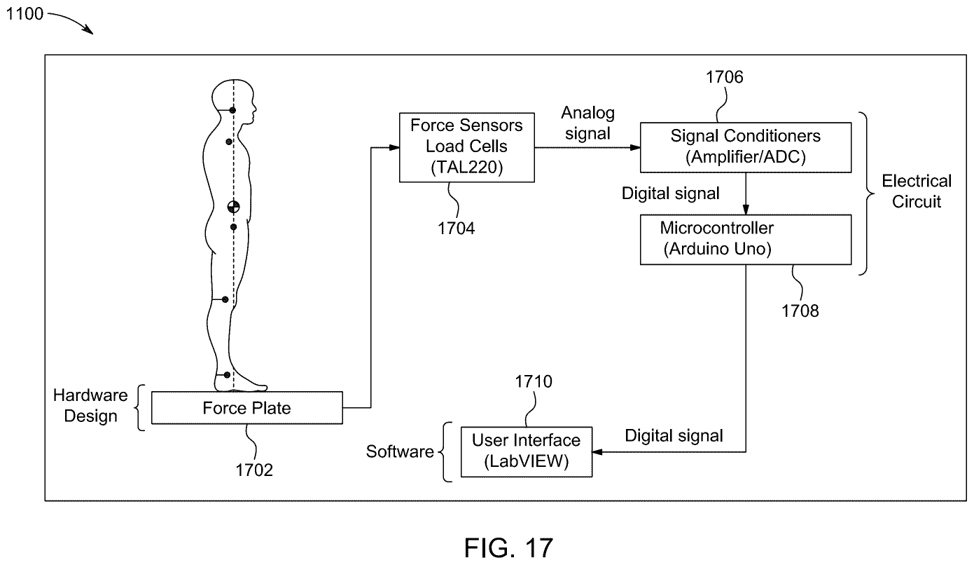

FIG. 17 shows a detailed block diagram of a force measurement platform 1700, according to some aspects. The force measurement platform 1700 may be an example of the force measurement platform 100.

As shown in FIG. 17, a mechanical load is applied to a surface of a force plate 1702 via a patient. Force sensors 1704 convert the mechanical load into electrical signals in an analog form. The electrical signals are conditioned and converted into digital signals by signal conditioners 1706. A microcontroller 1708 processes the amplified digital signal to collect all the data. The data is sent to a computing device 1710 for final processing.

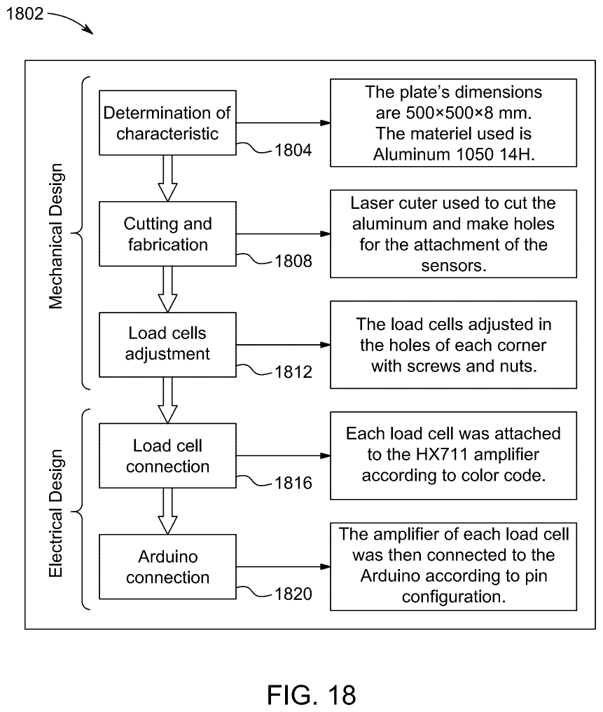

FIG. 18 describes a manufacturing process 1802 of the force measurement platform 100, according to aspects of the present disclosure.

As shown in FIG. 18, at step 1804, characteristics of components of the force measurement platform 100 were determined. In an example, a size of each of the surface plate and the base plate, and the material of the surface plate and the base plate were determined. For example, the size was determined to be 500×500×8 mm, and the material used was Aluminum 1050 15H. At step 1808, information regarding the fabrication and cutting of the surface plate and the base plate was determined. In an example, it was determined that the laser cutter is to be used to cut the aluminum and make holes for the attachment of load cells (i.e., the force sensors). At step 1812, load cells adjustment was determined. For example, it was determined that the load cells are to be adjusted in holes in each corner of the surface plate and/or the base with screws and nuts. At step 1816, connections of load cells were determined. In an example, it was determined that each load cell is to be attached to the HX711 amplifier (i.e., signal conditioner) according to color code. At step 1820, microcontroller (i.e., Arduino) connection was determined. For example, it was determined that the amplifier of each load cell is to be connected to the Arduino according to pin configuration.

In examples, the manufacturing of the force measurement platform 100 started with cutting the 8 mm Aluminum with laser cutter into two plates (a surface plate and a base plate) of 500×500 mm dimension, with the base plate having 2 (two) 5 mm diameter holes in each corner for the attachment of load cells.

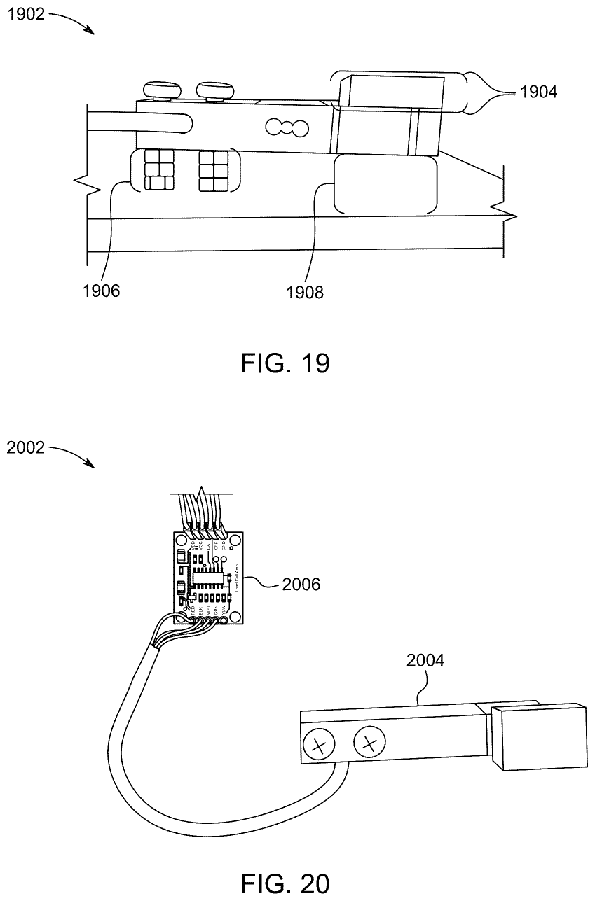

FIG. 19 shows an example 1902 of load cell adjustment, according to aspects of the present disclosure. Reference number 1904 represents added piece of wood that is used to make sure that the load cell is attached to the surface plate directly and can sense a load. Reference number 1906 represents added nuts to give space to other side of the load cell (represented by reference number “1908”).

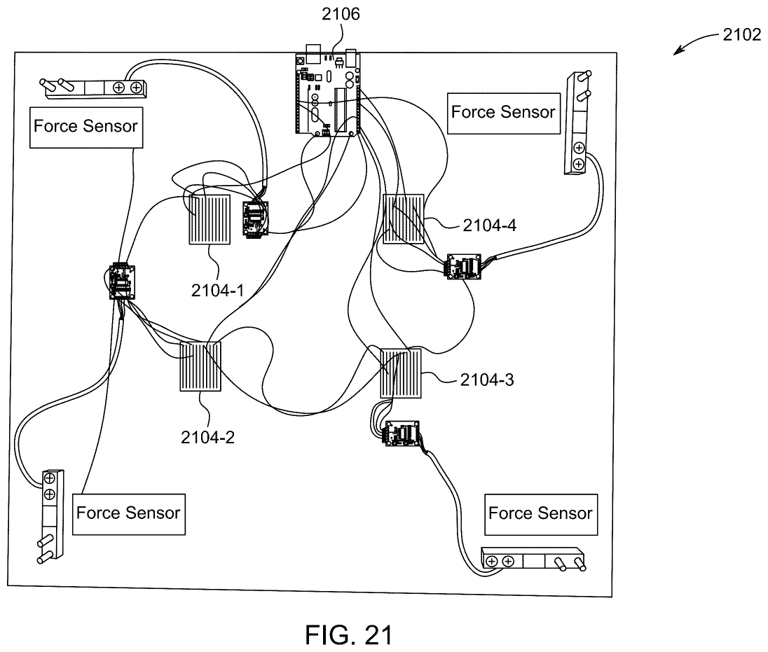

Referring back to FIG. 18, after the adjustment of the load cells, each loadcell was soldered to ensure proper connection to the left side of the HX711 amplifier according to Table 3 and as illustrated in FIG. 20. FIG. 20 illustrates an example 2002 of connections from load cell 2004 to HX711 amplifier 2006, according to aspects of the present disclosure.

TABLE 3
Connections for the load cell to HX711 amplifier

| Wire color | HX711 mode |
|---|---|
| Red | E+ (V_{CC}) |
| Black | E− (GND) |
| White | A+ (Output+) |
| Green | A− (Output−) |

Referring again to FIG. 18, the right side of each amplifier was connected to the Arduino. The connection from the amplifiers to the Arduino was made according to Table 4 provided below and is shown in FIG. 21.

TABLE 4
Connections from HX711 amplifier to Arduino

| | | |
|---|---|---|
| Wire color | HX711 node | Arduino |

Sensor 1

| | | |
|---|---|---|
| Black | GND | GND |
| Blue | DT | Pin (2) |
| Yellow | CLK | Pin (3) |
| White and orange | V_{CC} and V_{DD} | 5 V |

Sensor 2

| | | |
|---|---|---|
| Black | GND | GND |
| Blue | DT | Pin (4) |
| Yellow | CLK | Pin (5) |
| White and orange | V_{CC} and V_{DD} | 5 V |

Sensor 3

| | | |
|---|---|---|
| Black | GND | GND |
| Blue | DT | Pin (6) |
| Yellow | CLK | Pin (7) |
| White and orange | V_{CC} and V_{DD} | 5 V |

Sensor 4

| | | |
|---|---|---|
| Black | GND | GND |
| Blue | DT | Pin (8) |
| Yellow | CLK | Pin (9) |
| White and orange | V_{CC} and V_{DD} | 5 V |

FIG. 21 illustrates an example 2102 of connections between HX711 amplifiers and Arduino, according to aspects of the present disclosure. As shown in FIG. 21, each of HX711 amplifier 2101-1, HX711 amplifier 2101-2, HX711 amplifier 2101-3, and HX711 amplifier 2101-4 are connected to Arduino 2106.

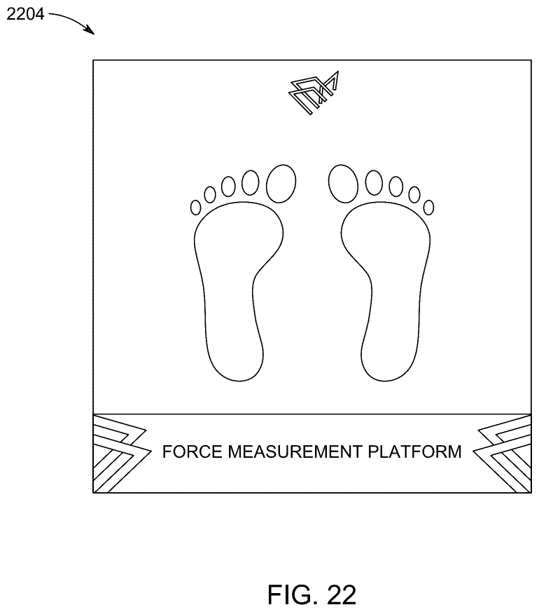

Referring again to FIG. 18, after completing the electrical connection, the surface plate and the base plate were adjusted together, and to ensure the adjustment, safety locks were added to each side of the force platform (force measurement assembly). A plastic sticker was used for covering and enhancing the force platform. FIG. 22 describes an example 2204 of the designed sticker for the force measurement assembly, according to aspects of the present disclosure. The sticker was designed with the real 64 dimensions of an average human foot to indicate where the patient (subject) should stand and place his/her foot in the force measurement assembly (force platform).

### EXAMPLES

The following examples describe and demonstrate exemplary embodiments of the force measurement platform, and the method of determining and monitoring postural stability, as described herein. The examples are provided solely for illustration and are not to be construed as limitations of the present disclosure, as many variations thereof are possible without departing from the spirit and scope of the present disclosure.

Examples of numerical modeling, calibration, and experimental setup are described below.

### Example 1: Numerical Modelling

As the subject stands on the force plate, the sensors may generate an analog signal that represents the GRF as a voltage. Using signal conditioning, the analog signal produced by each of the four sensors is converted into an amplified digital signal. Following that, the amplified digital signal enters a data acquisition device, which collects all the data and sends it to a software program for processing. Further, the forces data may be used in calculating and plotting COP_{AP} and COP_{ML} using Equations (2) and (3), which will be used to visualize the movement of the COP as COP sway. Finally, these results may be in calculating S_{TOT}, (using Equation (1)), A_{sway} using Equation (5), and V_{COP} (using Equation (4) or Equation (6) provided below)

$${V}_{COP} = \frac{{F}_{s}}{N} {\sum }_{k = 2}^{N} \sqrt{{( {COP}_{{AP}_{( i + 1 )}} - {COP}_{{AP}_{( i )}} )}^{2} + {( {COP}_{{ML}_{( i + 1 )}} - {COP}_{{ML}_{( i )}} )}^{2}} \qquad ( 6 )$$

### Example 2: Calibration

The calibration comprises removing any loads on the force measurement assembly and zeroizing the readings of the force measurement platform. The calibration further comprises placing a known mass on the force measurement assembly and running the force measurement platform. The calibration comprises recording a displayed mass, and calculating a calibration factor via a mathematical formula (Equation (7) provided below) and setting the calibration factor in the data acquisition and processing unit. The mathematical formula for calibration factor is provided below

$$Calibration factor = \frac{Capacity ( g )}{Sensitivity ( \frac{mV}{V} ) \times Excitation Voltage ( V )} \qquad ( 7 )$$

Arduino code was designed to calibrate the load cell sensors that have been used in the force measurement platform 100. Moreover, the Arduino code was used to ensure that the load cells operated smoothly, and that accurate measurements were acquired by applying specific weights. The Arduino code is used to calibrate each load cell by determining the zero and then applying known mass to the sensor. An example of the Arduino code for calibration of the load cell sensors is provided below.

```
#include <HX711_ADC.h>
#if defined(ESP8266)|| defined(ESP32) || defined(AVR)
#include <EEPROM.h>
#endif
//pins:
const int data1 = 4; // Sensor data
const int clock1 = 5; // Sensor clock
//HX711 identifies loadcell:
HX711_ADC Sensor(data1, clock1);
const int Val_Calib_Adress = 0;
unsigned long timing = 0;
void setup( ) {
  Serial.begin(57600); delay(10);
  Serial.println( );
  Serial.println(“Starting...”);
  Sensor.begin( );
  unsigned long time_stable = 2000;
  boolean for_tare = true;
  Sensor.start(time_stable, for_tare);
  if (Sensor.getTareTimeoutFlag( ) || Sensor.getSignalTimeoutFlag( )) {
    Serial.println(“Timeout! check HX711 to Arduino wiring and pin
configuration”);
      while (1);
    }
  else {
      Sensor.setCalFactor(1.0);
      Serial.println(“Starting Done”);
  }
  while (!Sensor.update( ));
  calibrate( ); // to start calibration
  }
  void loop( ) {
    const int activitySerial = 0; //to set the activity of the serial print
    static boolean searchNewdata = 0;
    // Searching for new data:
    if (Sensor.update( )) searchNewdata = true;
// obtain smoothed data from the dataset:
  if (searchNewdata) {
      if (millis( ) > timing + activitySerial) {
        float D = Sensor.getData( );
        Serial.print(“sensor output value: ”);
        Serial.println(D);
        searchNewdata = 0;
        timing = millis( );
      }
    }
  // serial monitor
  if (Serial.available( ) > 0) {
      char readserial = Serial.read( );
      if (readserial == ‘z’) Sensor.tareNoDelay( ); //perform tare
      else if (readserial == ‘c’) calibrate( ); // perform calibration
      else if (readserial == ‘d’) changeSavedCalFactor( ); //modify calibration factor
    }
    // tare completion
    if (Sensor.getTareStatus( ) == true) {
      Serial.println(“Tare done”);
    }
  }
  void calibrate( ) {
      Serial.println(“---”);
      Serial.println(“calibration started:”);
      Serial.println(“Put the sensor a smooth surface.”);
      Serial.println(“Remove loads from the sensor.”);
      Serial.println(“write ‘z’ to set the zero.”);
      boolean set = false;
      while (set == false) {
          Sensor.update( );
          if (Serial.available( ) > 0) {
            if (Serial.available( ) > 0) {
              char readserial = Serial.read( );
              if (readserial == ‘t’) Sensor.tareNoDelay( );
            }
          }
        if (Sensor.getTareStatus( ) == true) {
          Serial.println(“Tare Done”);
          set = true;
      }
      }
    Serial.println(“Now, put the known mass on the sensor.”);
    Serial.println(“Then write the weight of the mass (in grams).”);
    float mass = 0;
    set = false;
    while (set == false) {
        Sensor.update( );
        if (Serial.available( ) > 0) {
          mass = Serial.parseFloat( );
          if (mass != 0) {
            Serial.print(“The known mass: ”);
            Serial.println(mass);
            set = true;
          }
        }
        }
    Sensor.refreshDataSet( );
    float CalibVal_New = Sensor.getNewCalibration(mass); //set new calibration
factor
    Serial.print(“the New calibration factor is: ”);
    Serial.print(CalibVal_New);
    Serial.print(“Save it to EEPROM”);
    Serial.print(Val_Calib_Adress);
    Serial.println(“? y/n”);
      set = false;
      while (set == false) {
          if (Serial.available( ) > 0) {
              char readserial = Serial.read( );
              if (readserial == ‘y’) {
  #if defined(ESP8266)|| defined(ESP32)
              EEPROM.begin(512);
  #endif
              EEPROM.put(Val_Calib_Adress, CalibVal_New);
  #if defined(ESP8266)|| defined(ESP32)
            EEPROM.commit( );
  #endif
            EEPROM.get(Val_Calib_Adress, CalibVal_New);
            Serial.print(“Calibration factor ”);
          Serial.print(CalibVal_New);
          Serial.print(“is saved to EEPROM: ”);
          Serial.println(Val_Calib_Adress);
          set = true;
          }
          else if (readserial == ‘n’) {
              Serial.println(“calibration factor is not sent to EEPROM”);
              set = true;
            }
        }
      }
      Serial.println(“Calibration Ended”);
      Serial.println(“---”);
      Serial.println(“To re-calibrate, send ‘c’ from serial monitor.”);
      Serial. println(“For manual edit of the calibration value, send ‘d’ from serial
  monitor.”);
      Serial.println(“---”);
      }
      void changeSavedCalFactor( ) {
          float CalibVal_Old = Sensor.getCalFactor( );
          boolean set = false;
        Serial.println(“---”);
        Serial.print(“Current value is: ”);
        Serial.println(CalibVal_Old);
        Serial.println(“Now, send the new value”);
        float CalibVal_New;
        while (set == false) {
          if (Serial.available( ) > 0) {
              CalibVal_New = Serial.parseFloat( );
          if (CalibVal_New != 0) {
              Serial.print(“New calibration value is: ”);
              Serial.println(CalibVal_New);
              Sensor.setCalFactor(CalibVal_New);
              set = true;
            }
          }
        }
        set = false;
        Serial.print(“Save this value to EEPROM adress ”);
        Serial.print(Val_Calib_Adress);
        Serial.println(“? y/n”);
        while (set == false) {
          if (Serial.available( ) > 0) {
          char readserial = Serial.read( );
          if (readserial == ‘y’) {
      #if defined(ESP8266|| defined(ESP32)
            EEPROM.begin(512);
      #endif
            EEPROM.put(Val_Calib_Adress, CalibVal_New);
      #if defined(ESP8266)|| defined(ESP32)
            EEPROM.commit( );
      #endif
            EEPROM.get(Val_Calib_Adress, CalibVal_New);
            Serial.print(“Value ”);
            Serial.print(CalibVal_New);
            Serial.print(“ saved to EEPROM address: ”);
            Serial.println(Val_Calib_Adress);
            set = true;
            }
          else if (readserial == ‘N’) {
            Serial.println(“Value not saved to EEPROM”);
            set = true;
            }
          }
        }
      Serial.println(“End change calibration value”);
      Serial.println(“---”);
      }
```

The calibration procedure was done first by determining the zero, which was carried out with the Aluminum sheet on the top of load cell sensors, so that when the mass is applied to the Aluminum, it will start from the zero value. The known mass was then placed on the top of the Aluminum. The mass of the object was provided to the Arduino code by the user. Then the calibration factor was calculated. After that, measurements were read from the load cells by the code.

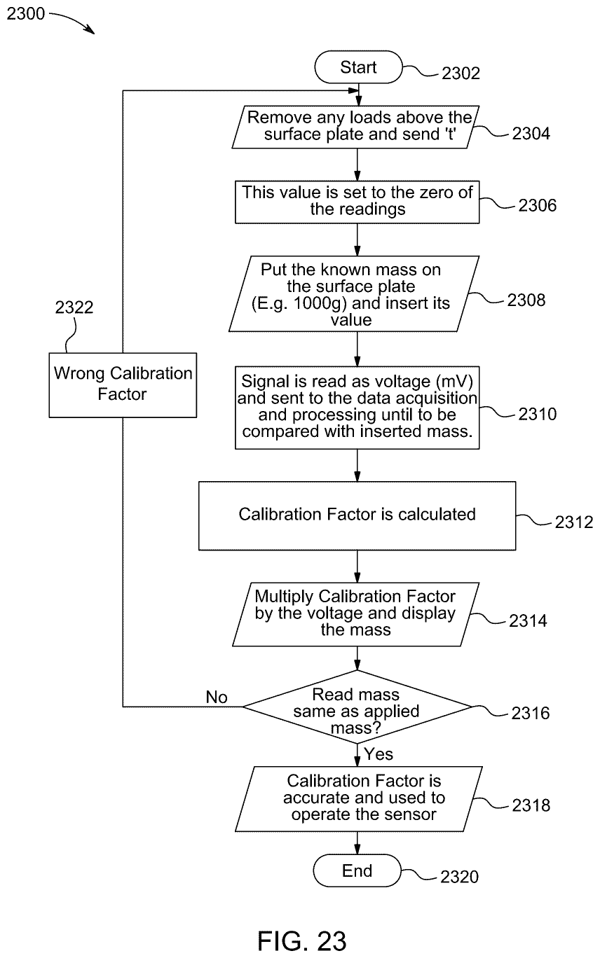

FIG. 23 describes a calibration process 2300, according to aspects of the present disclosure.

The calibration process starts at step 2302 of the calibration process 2300. At step 2304, loads are removed from above the surface plate (or the base plate) and “t” is sent. At step 2306, the value of the readings is set to the zero. At step 2308, unknown mass (for example of weight 1000 g) is put on the surface plate and its value is inserted. At step 2310, signal is read as voltage (mV) and sent to the Arduino to be compared with inserted mass. At step 2312, calibration factor is calculated. At step 2314, calibration factor is multiplied by the voltage and the mass is displayed. At step 2316, it was determined if the read mass is same as the applied mass. If the read mass is same as the applied mass, the calibration process 2300 proceeds to step 2318 (‘Yes’ branch). At step 2318, it is determined that the calibration factor is accurate and used to operate the sensor. At step 2320, calibration process 2300 ends. If the read mass is different from the applied mass, the calibration process 2300 proceeds to step 2322 (‘No’ branch). At step 2322, it is determined that the calibration factor is incorrect (wrong) and the calibration process 2300 starts again.

Many trials were carried out to find the exact calibration factors that give the most accurate measurement, which were found to be as shown in table 5.

TABLE 5
Calibration factors for each load cell

| Sensor Number | Calibration Factor |
|---|---|
| Load Cell 1 | 9.89 |
| Load Cell 2 | 7.72 |
| Load Cell 3 | 8.24 |
| Load Cell 4 | 7.55 |

### Example 3: Experimental Setup

The experiment was designed to examine and measure the GRF and COP to find the S_{TOT}, which was involved in the determination of posture stability. The components of the experiment included the force platform (or force measurement assembly), the data acquisition device (Arduino Uno) and the PC, where the force platform, the data acquisition device, and the PC were connected together. Steps involved the experiment are as follows.

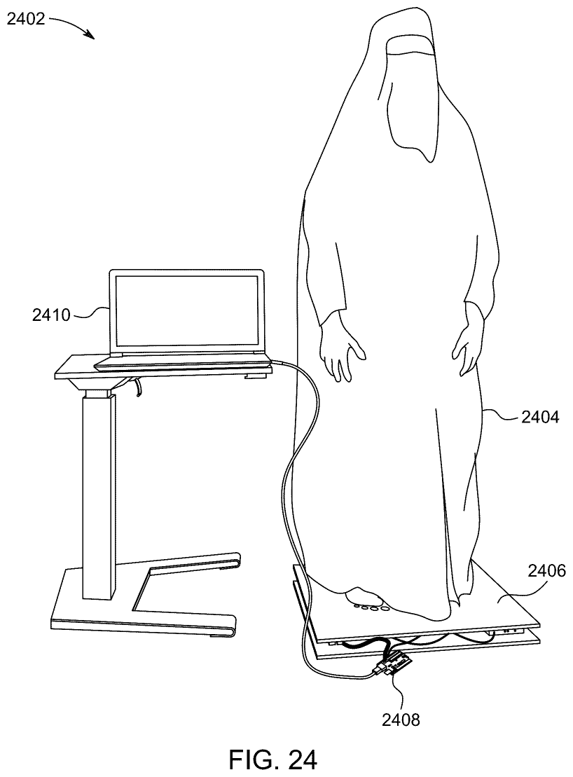

Step 1: A subject was asked to stand with bare feet on the force platform as shown in FIG. 24. FIG. 24 describes an experiment setup 2402 with a subject 2404, according to aspects of the present disclosure. As shown in FIG. 24, the subject 2404 is standing on the force platform 2406, where the force platform 2406 is connected to a data acquisition device 2408 and a PC 2410.

Step 2: The subject was asked to stand erect with his arms at the side of his body and to look forward at a specific point.

Step 3: The software program was run on the PC for “Eyes Open” test.

Step 4: The test continued reading the data for 20 seconds.

Step 5: The data acquisition device sent the data to the PC for processing.

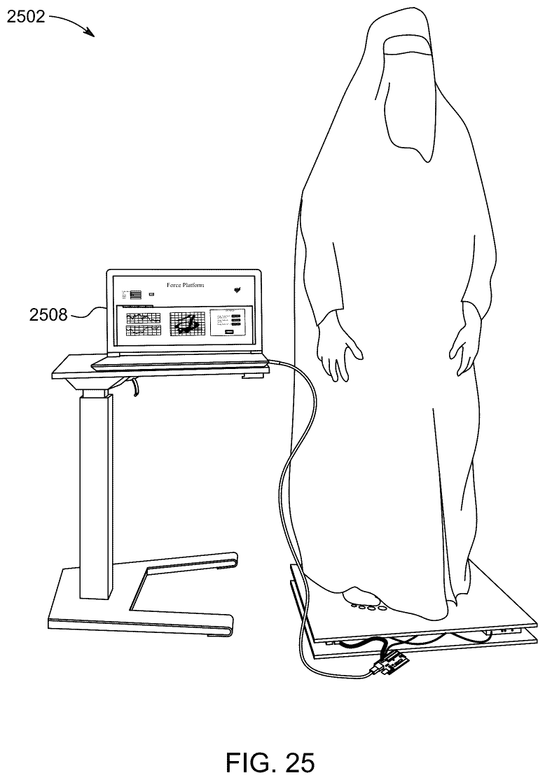

Step 6: The PC inputs the data into the software program to draw the sway path and calculate its parameters as shown in FIG. 25. FIG. 25 shows an experiment setup 2502 showing sway parameters, according to aspects of the present disclosure. As shown in FIG. 25, sway parameters are shown on PC 2508.

Step 7: The subject was asked to close his eyes and the software was run for the second test. Step 4, step 5, and step 6 are repeated with “Eyes Closed” test.

### Example 4: Analysis and Results

The signal of COP was used to analyze the influence of sensory information on the posture stability control. The position of the subject relative to the horizon was determined by the eyes, which assess the position of the body using 70 visual information acquired. On the other hand, the condition of the closed eye has limited sensory information derived from visual systems. When visual information is lost, instability related to vision loss may be distinguished from other sensory deficits. By combining the “Eyes Open” and “Eyes Closed” tests, the physician was able to diagnose balance defects and monitor the progress and efficiency of a treatment program.

Since many factors affected the postural sway, a normative database was used to compare the postural sway results of the subject being tested with a healthy peer as a reference. The used normative database was collected from 16,357 individuals, and it showed that the postural sway is mostly affected by the age and the gender of the person, and that the body shape has almost no effect on the sway. In an example, S_{TOT} was used as an indicator of the postural sway. It is used to separate the male data from the female data and divides each section into categories based on the age of the subject. For each category, there are percentile rankings that provide ranges of S_{TOT} to determine whether the patient is normal, at risk of getting balance abnormality, or abnormal. The ranges of the males and females are summarized in Table 6 and Table 7, respectively, provided below.

TABLE 6
S_{TOT} percentile ranking for male subjects

| Age | Percentile Ranking for S_{TOT} Results (cm) | | | | | | | | |
|---|---|---|---|---|---|---|---|---|---|
| Group | 10^{th} | 20^{th} | 30^{th} | 40^{th} | 50^{th} | 60^{th} | 70^{th} | 80^{th} | 90^{th} |
| 5-9 | 56 | 49 | 44 | 41 | 37 | 35 | 32 | 30 | 26 |
| 10-14 | 42 | 37 | 33 | 31 | 28 | 26 | 24 | 22 | 19 |
| 15-19 | 34 | 30 | 27 | 25 | 23 | 21 | 20 | 18 | 16 |
| 20-29 | 33 | 29 | 27 | 25 | 23 | 22 | 20 | 18 | 16 |
| 30-39 | 34 | 30 | 27 | 25 | 23 | 21 | 19 | 17 | 15 |
| 40-49 | 41 | 33 | 30 | 28 | 26 | 24 | 22 | 19 | 16 |
| 50-59 | 51 | 38 | 34 | 31 | 28 | 26 | 23 | 22 | 18 |
| 60-64 | 54 | 43 | 38 | 34 | 32 | 30 | 28 | 25 | 23 |
| 65-69 | 64 | 49 | 42 | 36 | 33 | 30 | 27 | 23 | 21 |
| 70-74 | 74 | 55 | 48 | 44 | 37 | 33 | 31 | 26 | 21 |
| 75-79 | 83 | 70 | 56 | 43 | 39 | 36 | 33 | 28 | 23 |
| 80-100 | 98 | 79 | 66 | 54 | 48 | 43 | 36 | 31 | 25 |

TABLE 7
S_{TOT} percentile ranking for female subjects

| Age | Percentile Ranking for S_{TOT} Results (cm) | | | | | | | | |
|---|---|---|---|---|---|---|---|---|---|
| Group | 10^{th} | 20^{th} | 30^{th} | 40^{th} | 50^{th} | 60^{th} | 70^{th} | 80^{th} | 90^{th} |
| 5-9 | 56 | 45 | 41 | 37 | 35 | 32 | 29 | 27 | 23 |
| 10-14 | 42 | 37 | 33 | 30 | 27 | 25 | 23 | 21 | 18 |
| 15-19 | 31 | 27 | 25 | 23 | 21 | 20 | 18 | 17 | 15 |
| 20-29 | 30 | 27 | 24 | 23 | 21 | 20 | 18 | 17 | 14 |
| 30-39 | 31 | 27 | 25 | 23 | 21 | 20 | 18 | 16 | 14 |
| 40-49 | 34 | 29 | 27 | 24 | 22 | 21 | 19 | 18 | 15 |
| 50-59 | 39 | 33 | 30 | 27 | 26 | 23 | 21 | 19 | 17 |
| 60-64 | 43 | 36 | 33 | 29 | 27 | 25 | 22 | 20 | 17 |
| 65-69 | 49 | 40 | 35 | 31 | 29 | 25 | 23 | 21 | 18 |
| 70-74 | 55 | 42 | 37 | 32 | 29 | 27 | 24 | 22 | 19 |
| 75-79 | 62 | 51 | 43 | 38 | 33 | 30 | 27 | 24 | 20 |
| 80-100 | 78 | 60 | 51 | 43 | 38 | 33 | 30 | 24 | 20 |

These ranges derived from the normative database were inserted into the block diagram of the developed LabVIEW software using a Case Structure function to classify them into males and females, and using a code written in a Formula Node to specify the age category to which the subject belongs. Few logic functions were then used to determine the health condition of the subject. An example of the code is provided below.

| Male: | |
|---|---|
| | int32 G; |
| | G=0; |
| | int32 y; |
| | if (A<=9) |
| | { |
| | if (P<=32) |
| | y=0; |
| | else if (P<=56) |
| | y=1; |
| | else y=2; |
| | 3 |
| | else if (A<=14) |
| | { |
| | if (P<=33) |
| | y=0; |
| | else if (P<=42) |
| | y=1; |
| | else y=2; |
| | } |
| | else if (A<=19) |
| | { |
| | if (P<=30) |
| | y=0; |
| | else if (P<=34) |
| | y=1; |
| | else y=2; |
| | } |
| | else if (A<=29) |
| | { |
| | if (P<=33) |
| | y=0; |
| | else y=2; |
| | } |
| | else if (A<=39) |
| | { |
| | if (P<=30) |
| | y=0; |
| | else if (P<=34) |
| | y=1; |
| | else y=2; |
| | } |
| | else if (A<=49) |
| | { |
| | if (P<=33) |
| | y=0; |
| | else if (P<=41) |
| | y=1; |
| | else y=2; |
| | } |
| | else if (A<=59) |
| | { |
| | if (P<=31) |
| | y=0; |
| | else if (P<=51) |
| | y=1; |
| | else y=2; |
| | } |
| | else if (A<=64) |
| | { |
| | if (P<=32) |
| | y=0; |
| | else if (P<=54) |
| | y=1; |
| | else y=2; |
| | } |
| | else if (A<=69) |
| | if (P<=33) |
| | y=0; |
| | else if (P<=64) |
| | y=1; |
| | else y=2; |
| | else if (A<=74) |
| | { |
| | if (P<=33) |
| | y=0; |
| | else if (P<=74) |
| | y=1; |
| | else y=2; |
| | } |
| | else if (A<=79) |
| | if (P<=33) |
| | y=0; |
| | else if (P<=83) |
| | y=1; |
| | else y=2; |
| | } |
| | else |
| | if (P<=31) |
| | y=0; |
| | else if (P<=98) |
| | y=1; |
| | else y=2; |
| | } |
| Female: | |
| | int32 G; |
| | G=1; |
| | int32 y; |
| | if (A<=9) |
| | { |
| | if (P<=29) |
| | y=0; |
| | else if (P<=56) |
| | y=1; |
| | else y=2; |
| | } |
| | else if (A<=14) |
| | if (P<=30) |
| | y=0; |
| | else if (P<=42) |
| | y=1; |
| | else y=2; |
| | } |
| | else if (A<=19) |
| | { |
| | if (P<=27) |
| | y=0; |
| | else if (P<=31) |
| | y=1; |
| | else y=2; |
| | } |
| | else if (A<=29) |
| | { |
| | if (P<=30) |
| | y=0; |
| | else y=2; |
| | else if (A<=39) |
| | if (P<=27) |
| | y=0; |
| | else if (P<=31) |
| | y=1; |
| | else y=2; |
| | } |
| | else if (A<=49) |
| | { |
| | if (P<=29) |
| | y=0; |
| | else if (P<=34) |
| | y=1; |
| | else y=2; |
| | } |
| | else if (A<=59) |
| | { |
| | if (P<=30) |
| | y=0; |
| | else if (P<=39) |
| | y=1; |
| | else y=2; |
| | } |
| | else if (A<=64) |
| | { |
| | if (P<=29) |
| | y=0; |
| | else if (P<=43) |
| | y=1; |
| | else y=2; |
| | } |
| | else if (A<=69) |
| | { |
| | if (P<=29) |
| | y=0; |
| | else if (P<=49) |
| | y=1; |
| | else y=2; |
| | } |
| | else if (A<=74) |
| | { |
| | if (P<=29) |
| | y=0; |
| | else if (P<=55) |
| | y=1; |
| | else y=2; |
| | } |
| | else if (A<=79) |
| | { |
| | if (P<=30) |
| | y=0; |
| | else if (P<=62) |
| | y=1; |
| | else y=2; |
| | } |
| | else |
| | if (P<=30) |
| | y=0; |
| | else if (P<=78) |
| | y=1; |
| | else y=2; |
| | } |

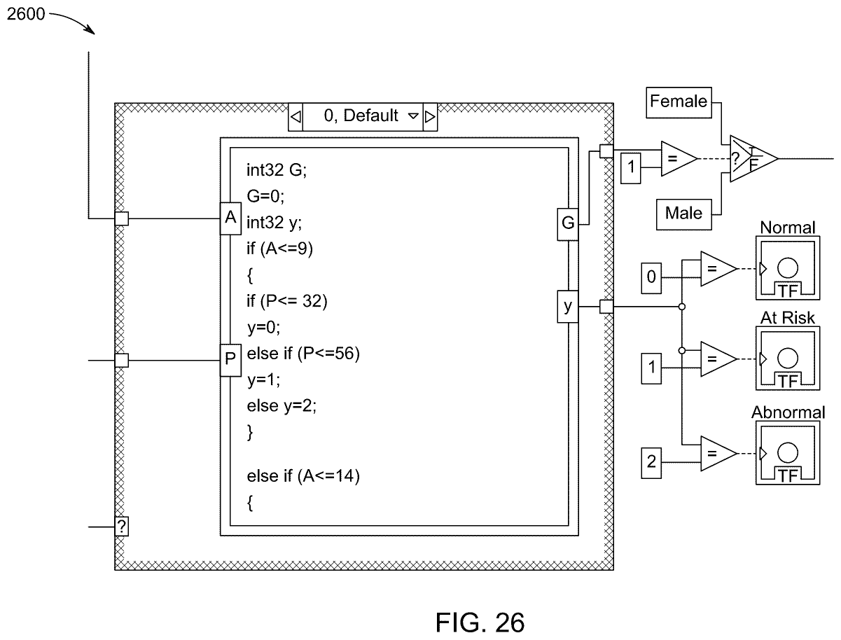

FIG. 26 shows an example of a normative database 2600, according to aspects of the present disclosure.

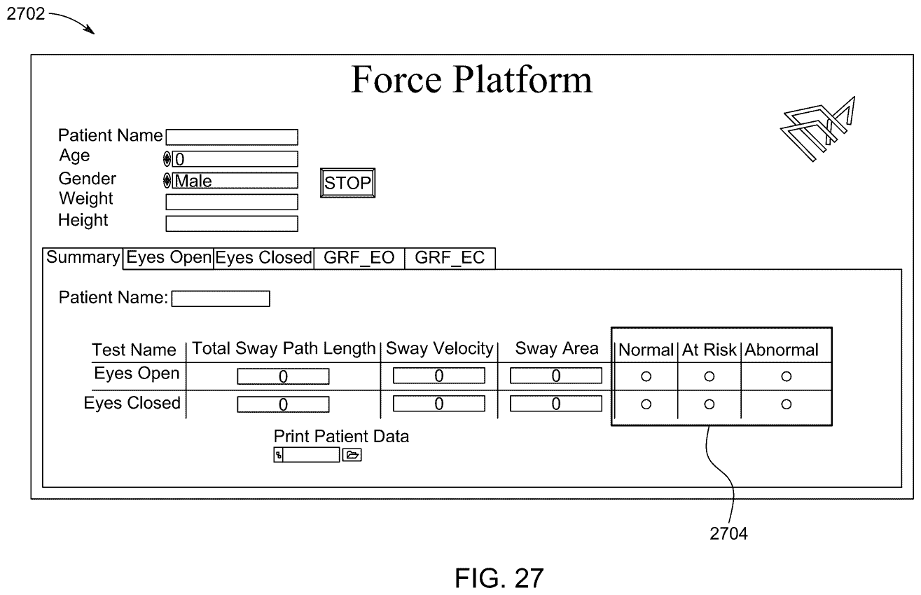

In order to make the program user-friendly and easier to use, Boolean LEDs were used to show the subject state for each test as shown in FIG. 27. FIG. 27 shows a front panel 2704 describing a state of a subject, according to aspects of the present disclosure. In FIG. 27, boolean LEDs (represented by number “2704”) were used to show the subject state for each test.

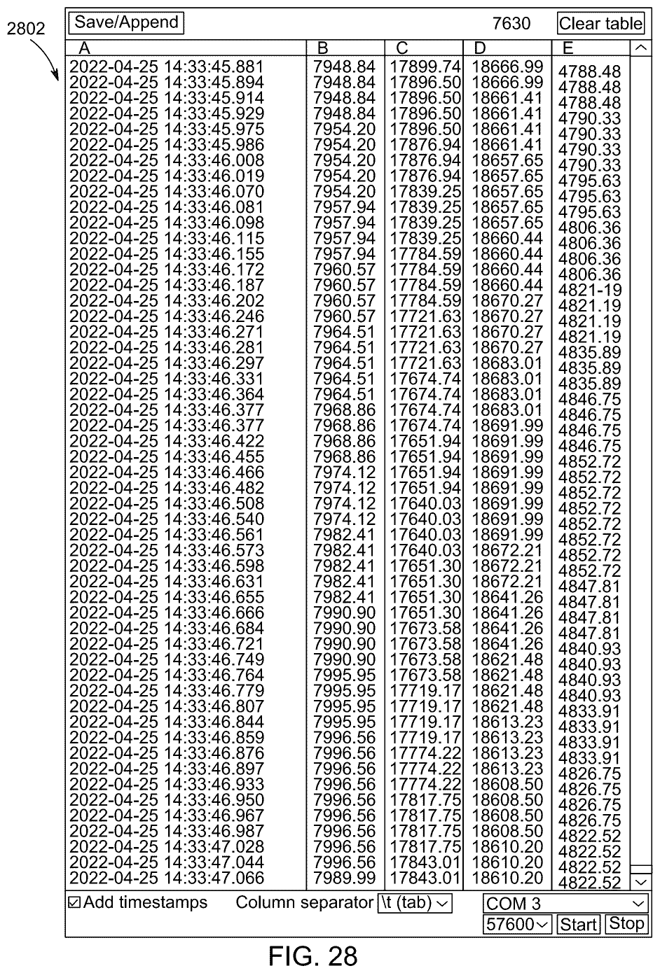

The force platform was intended to provide important data regarding the subject's stability to the physician. Each of the “Eyes Closed” test and the “Eyes Open” test were conducted for a duration of 20 seconds. Following calibration, the subject's force distribution resulting from his/her weight on the force platform was detected by the four sensors in each corner and displayed as raw data. FIG. 28 describes an example 2802 of data collected by sensors (force sensors), according to aspects of the present disclosure.

The raw data was then entered into the LabVIEW software program and processed using the Equations (1)-(6). After the data was processed, the data was displayed in five tabs including the “summary” tab, the “Eyes Open” tab, the “Eyes Closed” tab, the “GRF_EO” tab, and the “GRF_EC” tab on the front panel. As a result, the physician was enabled to obtain a detailed result of each test.

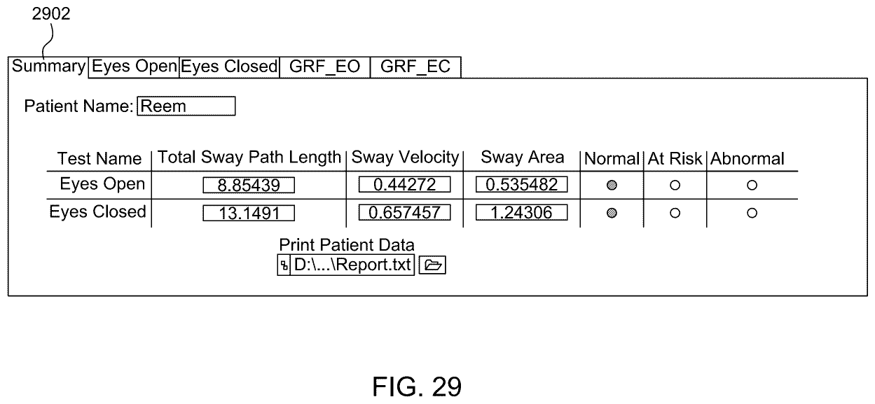

FIG. 29 shows the “summary” tab 2902 of the front panel, according to aspects of the present disclosure. The “summary” tab 2902 displayed a summary of the overall result. In an example, “summary” tab 2902 displayed an indicator of the subject's stability in both tests, i.e., whether it is normal, at risk, or abnormal.

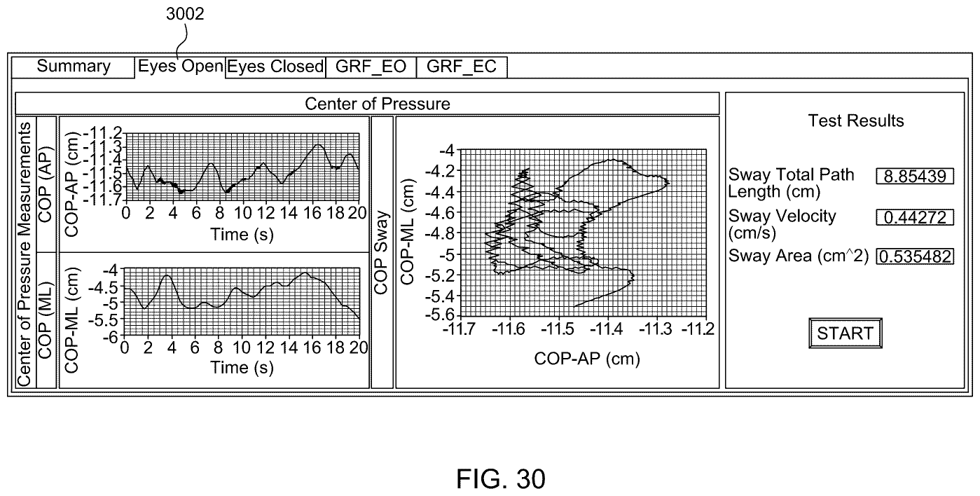

FIG. 30 shows the “Eyes Open” tab 3002 of the front panel, according to aspects of the present disclosure. The “Eyes Open” tab 3002 displays the detailed results of the “Eyes Open” test.

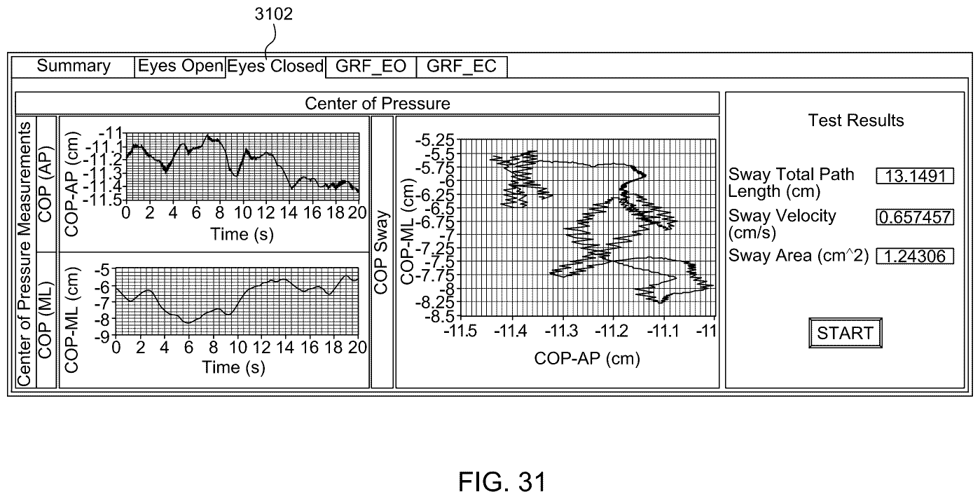

FIG. 31 shows the “Eyes Closed” tab 3102 of the front panel, according to aspects of the present disclosure. The “Eyes Closed” tab 3102 displays the detailed results of the “Eyes Closed” test. These results include COP_{AP} and COP_{ML} graphs, as well as S_{TOT}, V_{COP}, and A_{sway} values.

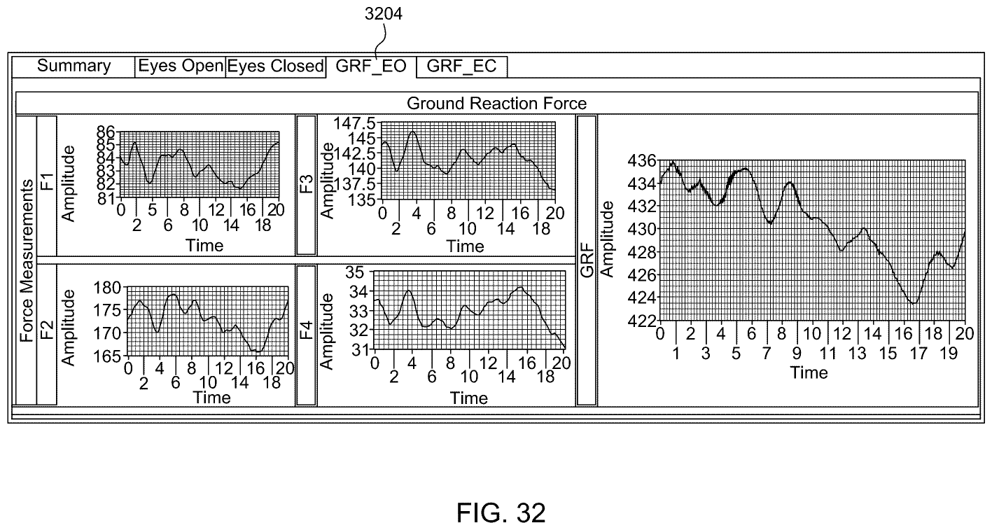

FIG. 32 shows the “GRF_EO” tab 3204 of the front panel, according to aspects of the present disclosure. The “GRF_EO” tab 3204 displays GRF in “Eyes Open” test.

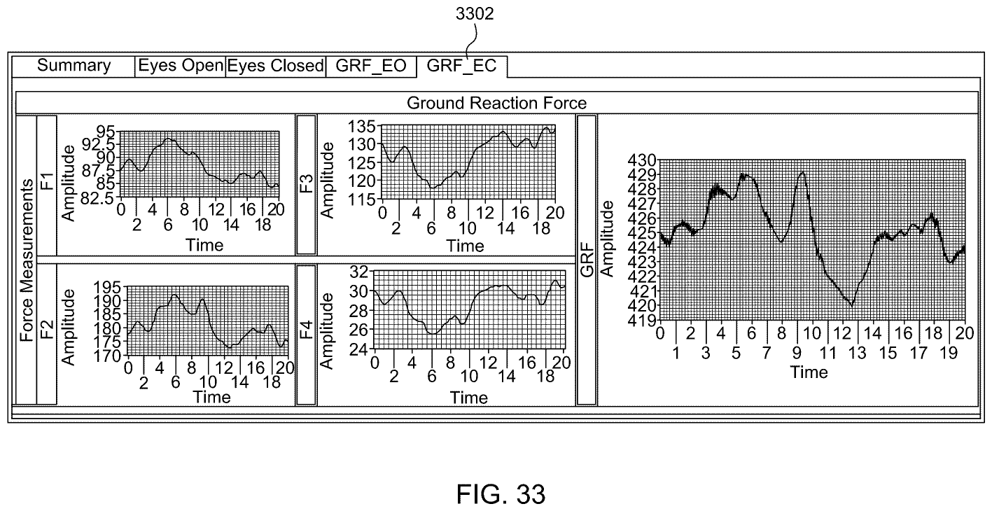

FIG. 33 shows the “GRF_EC” tab 3302 of the front panel, according to aspects of the present disclosure. The “GRF_EC” tab 3302 displays GRF in “Eyes Closed” test. Each sensor has its own graph displaying the GRF applied to it, as well as a graph demonstrating the sum of all forces.

According to an aspect, three validation tests were performed to ensure that the accuracy of results from the proposed prototype are comparable with the available product in the lab and manual calculations. Therefore, to ensure the validity of the force platform, several tests on the loadcell were performed such as resistance and voltage test, data verification test, and Data accuracy comparison test.

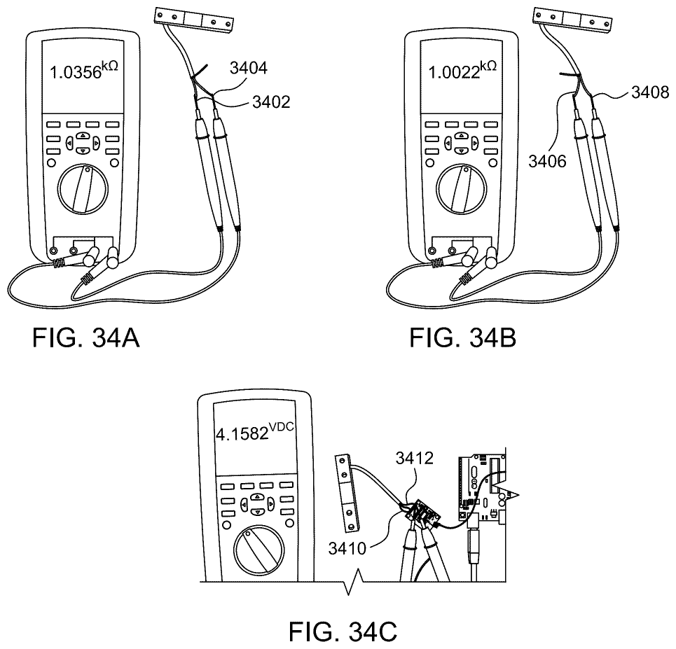

Resistance and voltage test was conducted for the validation of the loadcell, to compare between measured value and reference value from a data sheet. This helped to ensure that the sensors were working properly. The test started with checking accuracy of the input resistance and output resistance for each sensor. FIG. 34A shows loadcell sensor verification test for input resistance, according to aspects of the present disclosure. In FIG. 34A, wire 3402 represents black wire and wire 3404 represents red wire. FIG. 34B shows loadcell sensor verification test for output resistance, according to aspects of the present disclosure. In FIG. 34B, wire 3406 represents white wire and wire 3406 represents green wire. FIG. 34C shows loadcell sensor verification voltage test, according to aspects of the present disclosure. The voltage for each sensor was tested after connected with data acquisition device. In FIG. 34C, wire 3410 represents black wire and wire 3412 represents red wire. After completing the test, it was proven that the measured data almost matched the referenced data.

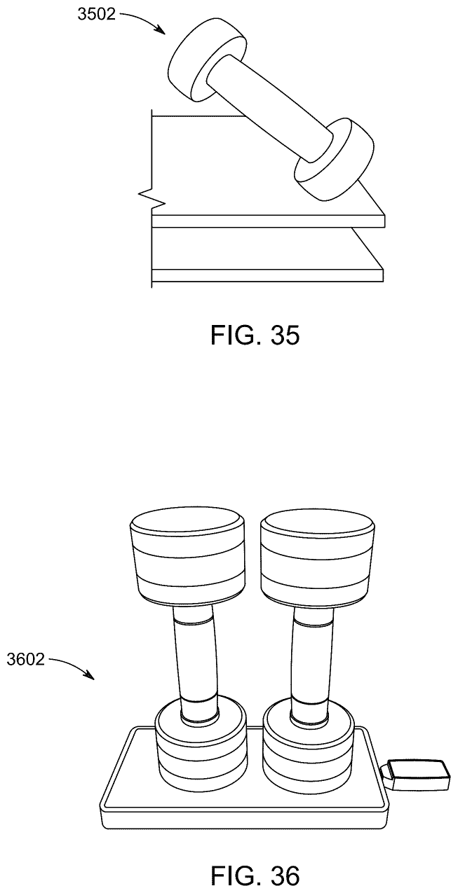

Data verification test was conducted to ensure the sensitivity of the sensor during any changes in angle that could result from COP sway. That is to ensure that the sensors can read the forces applied in all direction. The data verification test was performed by applying a mass of 1000 g at angle of 45° to make sure that the sensor could detect small changes in the applied angle. FIG. 35 shows a mass 3502 of 1000 g being applied at 45° on the force platform. In the data verification test, the data from the sensors was calibrated to measure the mass, and then Equation (8) provided below was used to convert it into GRF

$$GRF = m \times g = m \times 9.81 \qquad ( 8 )$$

In order to find the percent of error to calculate the accuracy of the force measurement platform 100, the calculated vertical GRF was found by using Equation (9) provided below.

$${GRF}_{y} = m \times 9.81 cos ( 45 ° ) \qquad ( 9 )$$

Table 8 provides a comparison of the measured and calculated force along with the percent of error.

TABLE 8
Accuracy measurement for each sensor

| | Measured | Calculated | |
|---|---|---|---|
| | Force | Force | % Error |
| Sensor 1 | 6936.72 N | 7245.86 N | $\frac{724586 - 6936.72}{74586} \times 100 = 4.3 \%$ |
| Sensor 2 | 6936.72 N | 7212.02 N | $\frac{721202 - 6936.72}{721202} \times 100 = 3.8 \%$ |
| Sensor 3 | 6936.72 N | 7432.35 N | $\frac{743235 - 6936.72}{743235} \times 100 = 6.6 \%$ |
| Sensor 4 | 6936.72 N | 7192.50 N | $\frac{719250 - 6936.72}{719250} \times 100 = 3.5 \%$ |

To ensure that the force measurement platform was working properly with highest accuracy, the force measurement platform 100 was compared to the force measurement platform available in the Biomechanics lab. As shown in the Table 9, the test carried with 10 kg dumbbells to measure the weight to ensure correct measurements.

TABLE 9
Accuracy of the force measurement platform 100
compared to Biomechanics lab force platform

| | Force measurement platform | | Biomechanics lab force |
|---|---|---|---|
| | prototype | | platform |
| Sensor 1 | 10.001 | kg | 10.1 kg |
| Sensor 2 | 9.999 | kg | |
| Sensor 3 | 9.993 | kg | |
| Sensor 4 | 10.000 | kg | |

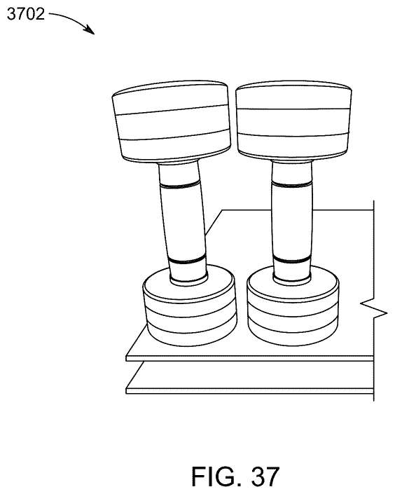

FIG. 36 shows an example 3602 of lab device test using 10 kg, according to aspects of the present disclosure. FIG. 37 shows an example 3702 of the force measurement platform 100 test (protype test) using 10 kg, according to aspects of the present disclosure.

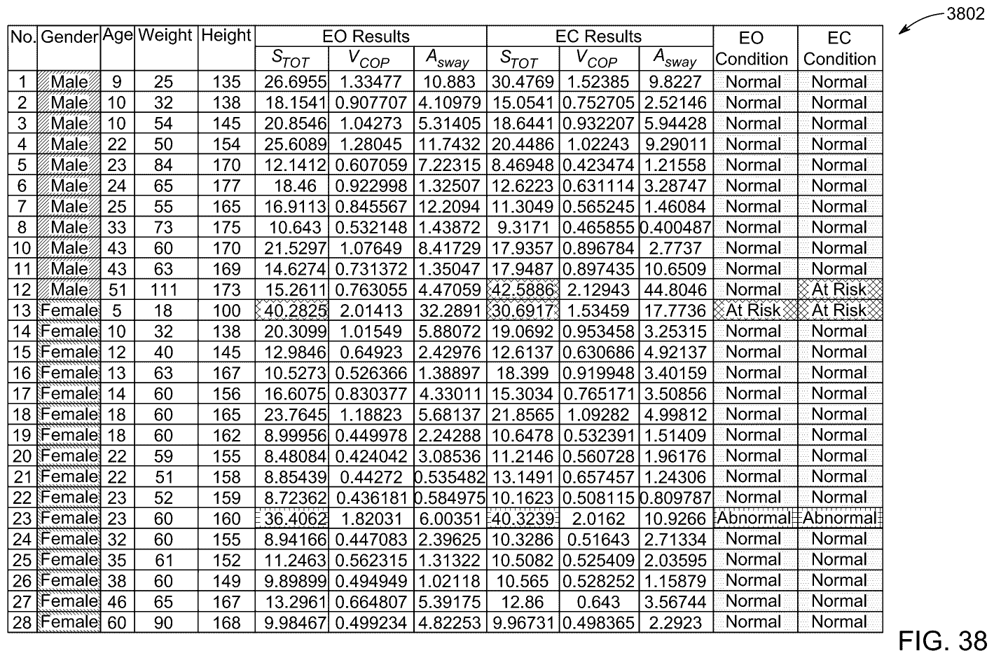

In examples, to verify whether the prototype met the goals of the present disclosure, number of trials had to be conducted. The trials included performing 30 experiments with 27 volunteers; 11 males and 16 females from different ages. The experiments were conducted, and the correct data was collected for each subject with his/her result. FIG. 38 shows an example 3802 of the data of various subjects, according to aspects of the present disclosure.

Before performing each experiment, the subject was asked about his medical history and whether there was any medical issue that could affect his balance. The examination was necessary in order to know what to expect, and in order to determine whether the results matched his history or not. Out of the 27 volunteers (subjects), only two had to repeat their experiments due to unrealistic results. The details about the wrong experiments and the percentage of error calculated using the number of experiments as shown in Table 10 and Table 11, respectively.

TABLE 10
Details of wrong trials

| | | Eyes Open Results | | | Eyes Closed Results | | |
|---|---|---|---|---|---|---|---|
| Subject | Experiment | S_{TOT} | V_{COP} | A_{SWAY} | S_{TOT} | V_{COP} | A_{SWAY} |
| 21 | Wrong | 23.0746 | 1.15373 | 3.71488 | 30.1324 | 1.50662 | 8.47179 |
| | Correct | 8.85439 | 0.44272 | 0.535482 | 13.1491 | 0.657457 | 1.24306 |
| 22 | Wrong | 83.2206 | 4.16103 | 161.846 | 85.276 | 4.2638 | 74.686 |
| | Correct | 8.72362 | 0.436181 | 0.584975 | 10.1623 | 0.508115 | 0.809787 |

TABLE 11
Percentage of experimental error

| | Total | Correct | Wrong |
|---|---|---|---|
| Number of | 29 | 27 | 2 |
| experiments | | | |

| | |
|---|---|
| Percentage of Error | $\frac{2}{29} \times 100 = 6.9 \%$ |

The results show that most subjects were normal, as most of them were healthy volunteers with no health conditions that could affect their stability. The summary of their data and the average of their results are shown in Table 12 provided below.

TABLE 12
Summary of subject data

| | Eyes Open Results | | | Eyes Closed Results | | |
|---|---|---|---|---|---|---|
| Gender | S_{TOT} | V_{COP} | A_{SWAY} | S_{TOT} | V_{COP} | A_{SWAY} |
| Male | 18.2624 | 0.9131 | 6.2258 | 18.6191 | 0.9310 | 8.3793 |
| Female | 15.5818 | 0.7791 | 4.9623 | 16.1038 | 0.8052 | 4.1210 |

Away from the healthy subject, there were three subjects whose results were not normal. Subject “12”, a male (51 years old) with 111 kg body weight and 173 cm height, was suffering from chronic Tinnitus, which is characterized by ringing sound in the ear. When the Tinnitus continues for a long period, it can lead to a loss of focus which often results in losing balance. This was observed during the test, where the subject “12” was able to stand quietly for the first 20 seconds while his eyes were open, with S_{TOT} of 15.2611 cm which is considered to be normal in comparison with the normal S_{TOT} of his age group as shown in Table 13 provided below.

TABLE 13
S_{TOT} of Subject “12”

| | | | | Age | Age | Age |
|---|---|---|---|---|---|---|
| | | | | group | group | group |
| | | | | Normal | ‘risky’ | Abnormal |
| Gender | Age | Test | S_{TOT} | S_{TOT} | S_{TOT} | S_{TOT} |
| Male | 51 | Eyes Open | 15.2611 | S_{TOT} < 31 | 31 < S_{TOT} < 51 | S_{TOT} > 51 |
| | | Eyes Closed | 42.5886 | | | |

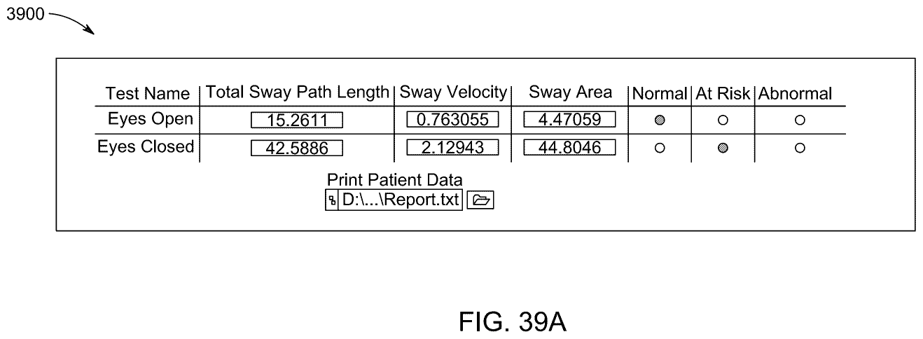

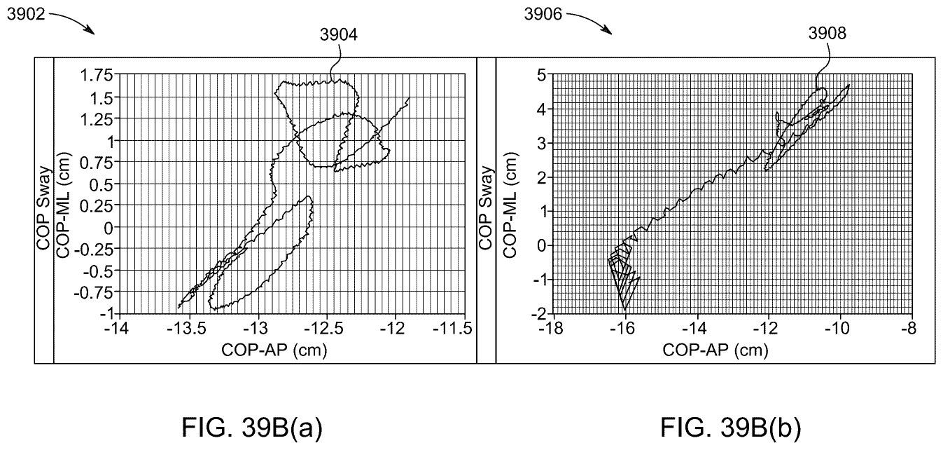

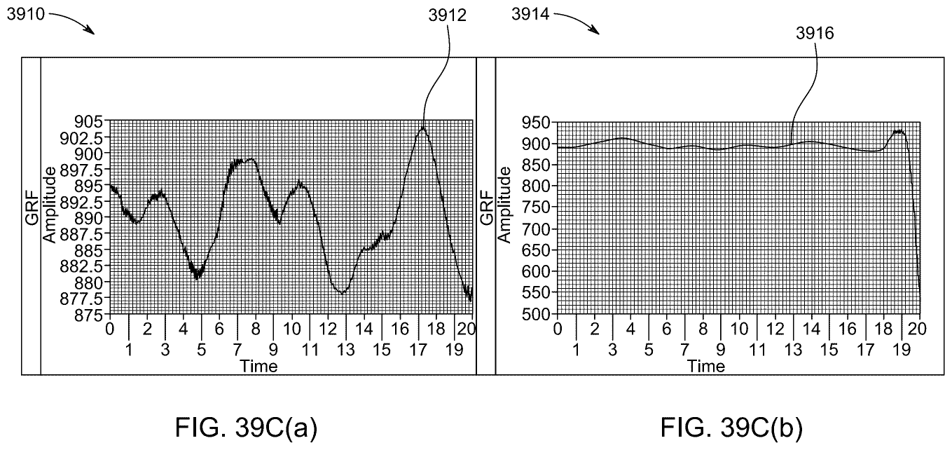

However, during the “Eyes Closed” test, results of subject “12” showed sharp decrease in the stability and increase in the S_{TOT} to reach 42.5886 cm. This result, according to the database, did not indicate an abnormal condition, rather it suggested that the subject “12” is at risk of losing stability during standing if his condition was not treated. The test results of subject “12” from the program are shown in FIG. 39A, FIG. 39B(a), FIG. 39B(b), FIG. 39C(a), and FIG. 39C(b). In particular, FIG. 39A shows an example 3900 of sway parameters for subject “12”, according to aspects of the present disclosure. FIG. 39B(a) shows an example 3902 of COP sway representation 3904 for “Eyes Open” test for subject “12”, according to aspects of the present disclosure. FIG. 39B(b) shows an example 3906 of COP sway representation 3908 for “Eyes Closed” test for subject “12”, according to aspects of the present disclosure. FIG. 39C(a) shows an example 3910 of total GRF 3912 for “Eyes Open” test, according to aspects of the present disclosure. FIG. 39C(b) shows an example 3914 of COP sway representation 3916 for “Eyes Closed” test for subject “12”, according to aspects of the present disclosure.

In examples, subject “13”, a female (5 years old) with a body weight of 18 kg and height of 100 cm, was suffering from Usher syndrome with moderate hearing loss and vision problems from the time of birth. The type of hearing loss associated with this syndrome is sensorineural, which means that it is caused by an abnormality in the inner ear which is responsible for maintaining balance. As a result, this condition is usually associated with balance problems during standing and walking. The results of the examination proved this conclusion, it showed that S_{TOT} of both “Eyes Open” test and “Eyes Closed” test were out of the normal range in comparison with S_{TOT} of her age group as shown in Table 14 provided below.

TABLE 14
S_{TOT} of Subject “13”

| | | | | Age | Age | Age |
|---|---|---|---|---|---|---|
| | | | | group | group | group |
| | | | | Normal | ‘risky’ | Abnormal |
| Gender | Age | Test | S_{TOT} | S_{TOT} | S_{TOT} | S_{TOT} |
| Female | 5 | Eyes Open | 40.2825 | S_{TOT} < 29 | 29 < S_{TOT} < 56 | S_{TOT} > 56 |
| | | Eyes Closed | 30.6917 | | | |

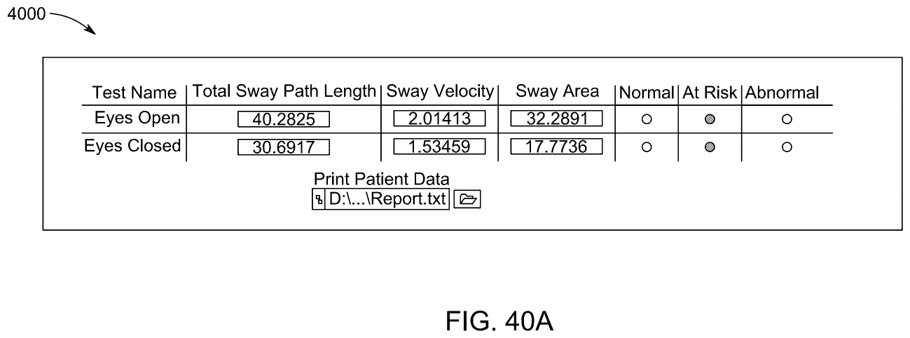

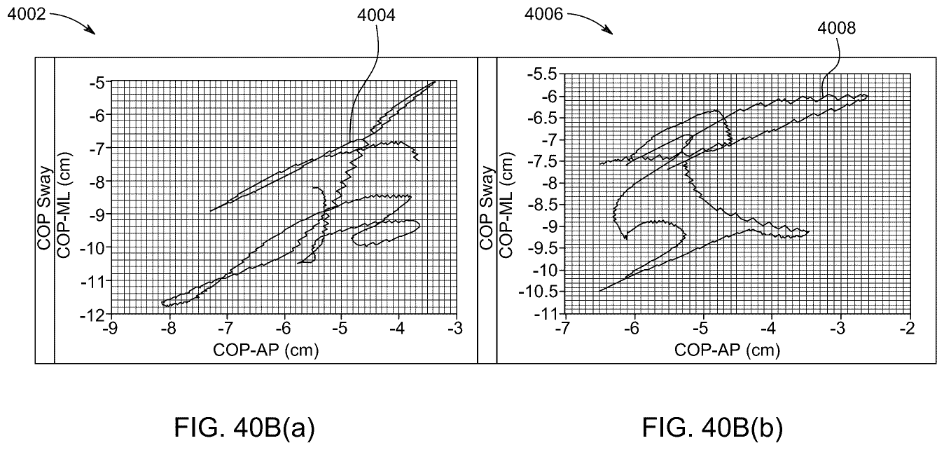

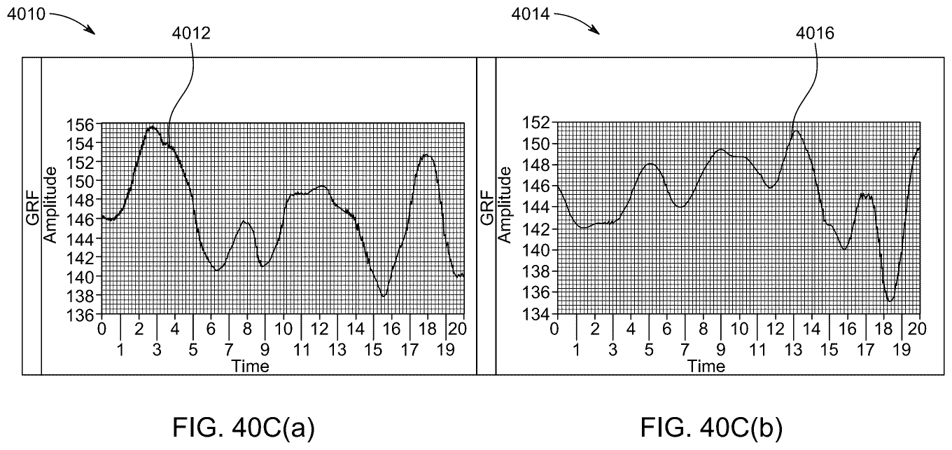

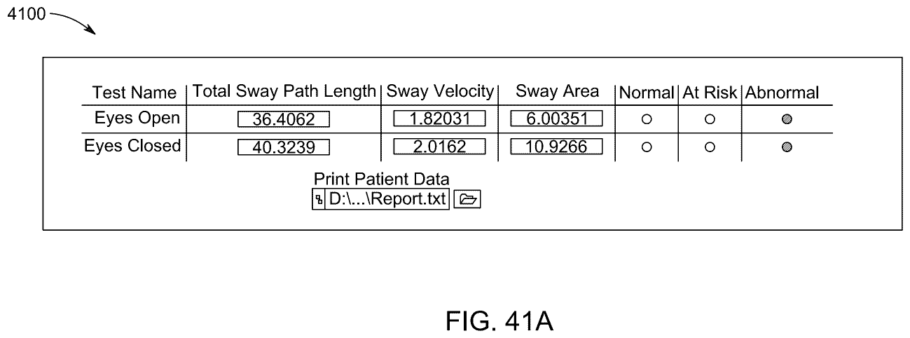

However, it is also indicated that the subject “13” is still within the ‘risky’ range, and that she still could stand upright with minimal assistance. The other observation noted while reviewing the results is that her S_{TOT} during “Eyes Closed” test was less than “Eyes Open” test with almost 10 cm difference, which is common in her age group as young children can get distracted easily by the surrounding while their eyes are open. The test results of subject “13” from the program are shown in FIG. 40A, FIG. 40B(a), FIG. 40B(b), FIG. 40C(a), and FIG. 40C(b). In particular, FIG. 40A shows an example 4000 of sway parameters of subject “13”, according to aspects of the present disclosure. FIG. 40B(a) shows an example 4002 of COP sway representation 4004 for “Eyes Open” test of subject “13”, according to aspects of the present disclosure. FIG. 40B(b) shows an example 4006 of COP sway representation 4008 for “Eyes Closed” test of subject “13”, according to aspects of the present disclosure. FIG. 40C(a) shows an example 4010 of total GRF 4012 for “Eyes Open” test of subject “13”, according to aspects of the present disclosure. FIG. 40C(b) shows an example 4014 of COP sway representation 4016 for “Eyes Closed” test of subject “13”, according to aspects of the present disclosure.

Subject “23”, a female (23 years old) with 60 kg body weight and 160 cm height that suffers from a neuromuscular scoliosis, which is a condition that curves and twists the spine to the side. Patient with scoliosis usually experience progressive loss of static balance during standing and sitting which can be easily detected during COP sway test. During both “Eyes Open” test and “Eyes Closed” test, the subject “23” could barely stand, and her results showed clear imbalance in both tests. The S_{TOT} of each test were 36.4062 and 40.3239 cm in “Eyes Open” test and “Eyes Closed” test respectively, which both are considered abnormal when compared to the normal S_{TOT} of her age group which is 30 cm as indicated in Table 15 provided below.

TABLE 15
S_{TOT} of Subject “23”

| | | | | Age | Age | Age |
|---|---|---|---|---|---|---|
| | | | | group | group | group |
| | | | | Normal | ‘risky’ | Abnormal |
| Gender | Age | Test | S_{TOT} | S_{TOT} | S_{TOT} | S_{TOT} |
| Female | 23 | Eyes Open | 36.4062 | S_{TOT} < 30 | — | S_{TOT} > 30 |
| | | Eyes Closed | 40.3239 | | | |

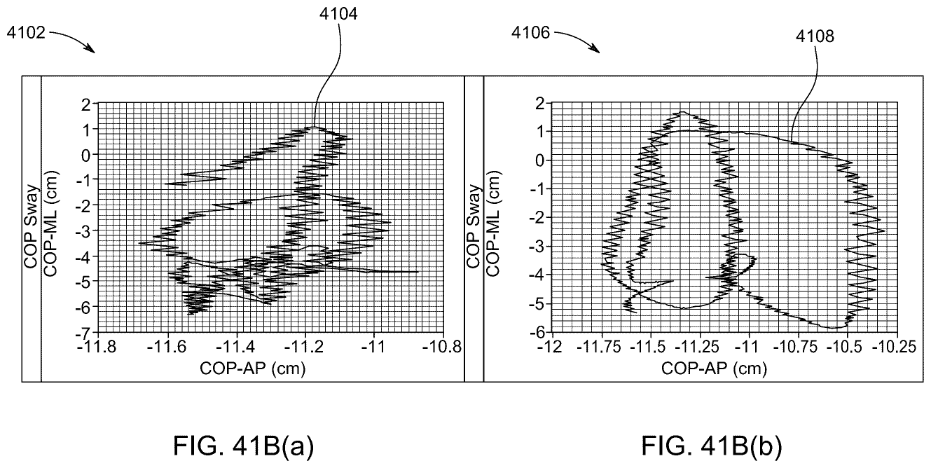

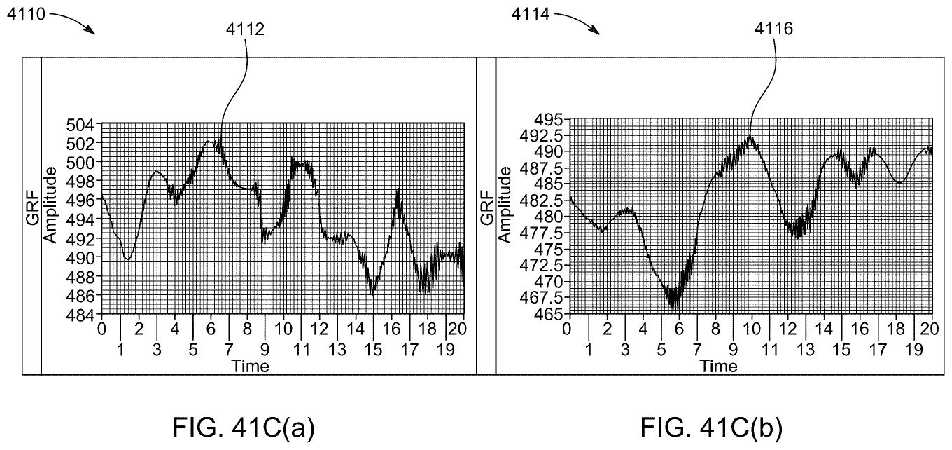

The results of subject “23” from the program are shown in FIG. 41A, FIG. 41B(a), FIG. 41B(b), FIG. 41C(a), and FIG. 41C(b).

In particular, FIG. 41A shows an example 4100 of sway parameters of subject “23”, according to aspects of the present disclosure. FIG. 41B(a) shows an example 4102 of COP sway representation 4104 for “Eyes Open” test of subject “23”, according to aspects of the present disclosure. FIG. 41B(b) shows an example 4106 of COP sway representation 4108 for “Eyes Closed” test of subject “23”, according to aspects of the present disclosure. FIG. 41C(a) shows an example 4110 of total GRF 4112 for “Eyes Open” test of subject “23”, according to aspects of the present disclosure. FIG. 41C(b) shows an example 4114 of COP sway representation 4116 for “Eyes Closed” test of subject “23”, according to aspects of the present disclosure.

In the present disclosure, Aluminum was chosen for the fabrication of the force platform due to its features among other materials including the light weight and high sensitivity with the load cells. The specific type of Aluminum selected was alloy 1050 14H, The thickness of 8 mm Aluminum sheet was chosen to attain a high capacity to hold the human body. The physical and mechanical properties of alloy 1050 14H-8 mm are provided in Table 16 and Table 17, respectively.

TABLE 16
Physical Properties of Aluminum alloy 1050

| Physical Property | Value | |
|---|---|---|
| Density | 2.71 | g/cm³ |
| Melting Point | 650° | C. |
| Thermal Expansion | 24 × 10−6/K | |
| Modulus of Elasticity | 71 | GPa |
| Thermal Conductivity | 222 | W/m · K |
| Electrical Resistivity | 0.0282 × 10−6 | Ω · m |

TABLE 17
Mechanical Property of Aluminum alloy 1050

| Mechanical Property | Value | |
|---|---|---|
| Tensile Strength | 105-145 | MPa |
| Proof Stress | 85 | Min MPa |
| Hardness Brinell | 34 | HB |
| Elongation A | 12 | Min % |

The total cost of prototype design around 430 U.S. Dollars as shown in Table 18, which lists all expenses of the project

TABLE 18
Project Cost Analysis

| | | Unit Price (U.S. | Cost (U.S. |
|---|---|---|---|
| Components | Quantity | Dollar) | Dollars) |
| Aluminum Sheet | 2 | 72 | 144 |
| Load Cell sensor | 4 | 30 | 120 |
| (TAL220) | | | |
| HX711 Amplifier | 4 | 10.95 | 43.8 |
| Arduino UNO | 1 | 39.7 | 39.7 |
| Electronics | many | 59.7 | 59.7 |
| Screws and Hex | Many | 5.9 | 5.9 |
| Sticker | 1 | 13 | 13 |
| | | | Total Cost 426.1$ |

Table 19 compares between the design of the force measurement platform 100 and conventional force measurement platforms. The force measurement platform 100 was found to be with the lowest cost, suitable dimensions and acceptable measuring range

TABLE 19
Comparison between the force measurement platform
100 and conventional force measurement platforms

| | | Dimensions | Measuring | Cost (U.S. |
|---|---|---|---|---|
| Force Plate | Sensor type | (mm) | range (kN) | Dollars) |
| Force | Load cells | 500 × 500 × | Up to 1.96 | 453.12 |
| measurement | | 43.7 | | |
| platform 100 | | | | |
| Conventional | Load cells | 502 × 502 × | Up to 1.112 | 14,900 |
| Force | | 45.47 | | |
| measurement | | | | |
| platform - AMTI | | | | |
| Conventional | Piezoelectric | 600 × 400 × | Up to 10 | 10,000 |
| Force | crystal | 35 | | |
| measurement | | | | |
| platform - Kistler | | | | |

The force measurement platform 100 may be used in safety tests to prevent injuries during athlete's performance assessments before competitions, or to test the safety of the lower limb prosthesis. The force measurement platform 100 may be used to measure workplace safety and ergonomics. Moreover, the force measurement platform 100 is classified as class I medical device according to the FDA, because it has lower risk comparing to another medical device as it is completely non-invasive. The force measurement platform 100 results in a continuous improvement of healthcare service in public health facilities, thereby lowering the cost of treatment while improving the quality of care. This will result in societal benefits and user satisfaction. Also, the force measurement platform 100 may improve overall health worldwide since it is affordable and has high accuracy.

While various embodiments of the methods and systems have been described, these embodiments are illustrative and in no way limit the scope of the described methods or systems. Those having skill in the relevant art can effect changes to form and details of the described methods and systems without departing from the broadest scope of the described methods and systems. Thus, the scope of the methods and systems described herein should not be limited by any of the illustrative embodiments and should be defined in accordance with the accompanying claims and their equivalents.

1: A force measurement platform, comprising:
a force measurement assembly, comprising:
a surface plate having a first side and a second side opposite to the first side;
a base plate having a first side and a second side opposite to the first side;
wherein the second side of the surface plate is above and opposite to the first side of the base plate;
a plurality of force sensors;
wherein the plurality of force sensors are mutually diagonally arranged in four quadrants of the force measurement assembly with each quadrant containing at least one force sensor;
an optional secondary force sensor located in a geometric center of the force measurement assembly;
wherein the surface plate is supported on the base plate via the plurality of force sensors and the optional secondary force sensor;
a data acquisition and processing unit, comprising:
a plurality of signal conditioners;
a microcontroller;
a computing device;
wherein the computing device has a communications interface coupled to the data acquisition and processing unit; and
wherein the force measurement platform removably retains the computing device.

2: The force measurement platform of claim 1, wherein the force sensor comprises:
a beam portion having a first end and a second end;
wherein the first end of the beam portion is connected to the second side of the surface plate around at least one first pivot axis that is perpendicular to the surface plate, and is configured to receive at least a portion of a load that is applied to the surface plate;
wherein the second end of the beam portion is mounted to the first side of the base plate around at least one second pivot axis that is perpendicular to the base plate;
wherein the at least one first pivot axis is parallel to the at least one second pivot axis; and
a plurality of deformation sensing elements disposed on the beam portion of the force sensor along a longitudinal axis between the first end and the second end of the beam portion, and wherein the plurality of deformation sensing elements can covert an input mechanical load to an electrical signal representative of the load that is applied to the surface plate.

3: The force measurement platform of claim 1, wherein the optional secondary force sensor comprises:
a beam portion having a first end and a second end;
wherein the first end of the beam portion is connected to the second side of the surface plate around at least one first pivot axis that is perpendicular to the surface plate, and is configured to receive at least a portion of a load that is applied to the surface plate;
wherein the second end of the beam portion is mounted to the first side of the base plate around at least one second pivot axis that is perpendicular to the base plate;
wherein the at least one first pivot axis is parallel to the at least one second pivot axis; and
a plurality of deformation sensing elements disposed on the beam portion of the force sensor along a longitudinal axis between the first end and the second end of the beam portion, and wherein the plurality of deformation sensing elements can covert an input mechanical load to an electrical signal representative of the load that is applied to the surface plate.

4: The force measurement platform of claim 1, wherein the force sensor is at least one selected from the group consisting of a capacitive displacement sensor, an inductive force sensor, a strain gauge, a piezoelectric force sensor, a force sensing resistor, a piezoresistive force sensor, a thin film force sensor, and a quantum tunnelling composite-based force sensor.

5: The force measurement platform of claim 1, wherein the optional secondary force sensor is at least one selected from the group consisting of a capacitive displacement sensor, an inductive force sensor, a strain gauge, a piezoelectric force sensor, a force sensing resistor, a piezoresistive force sensor, a thin film force sensor, and a quantum tunnelling composite-based force sensor.

6: The force measurement platform of claim 1, wherein:
each of the plurality of signal conditioners is operatively connected to each of the plurality of force sensors and the optional secondary force sensor of the force measurement assembly; and
the plurality of signal conditioners are operatively connected to the microcontroller.

7: The force measurement platform of claim 1, wherein the plurality of signal conditioners comprises at least one amplifier, at least one high-pass filter, and at least one low-pass filter.

8: The force measurement platform of claim 1, wherein the microcontroller comprises a memory for saving or installing an application program, or a software program, and wherein the application program or the software program from an internet or a cloud server is downloaded for interpreting and executing digital signals.

9: The force measurement platform of claim 1, wherein the data acquisition and processing unit is configured to receive one or more signals that are representative of a load being applied to a surface of the force measurement assembly and to record a location of the applied load on the surface of the force measurement assembly by computing a center of pressure (COP) for the applied load as determined by ASTM F3109-16.

10: The force measurement platform of claim 9, wherein the data acquisition and processing unit is further configured to monitor the signals of the applied load over time to visualize a sway or a change in location of the load being applied on surface of the force measurement assembly by computing the COP.

11: The force measurement platform of claim 1, wherein the computing device:
presents a data visualization configuration interface according to a load applied to the force measurement platform; and
comprises a personal computer (PC), a server, a mobile computing device, and a computing circuit.

12: The force measurement platform of claim 1, wherein both the surface plate and the base plate are made of an aluminum alloy that is composed of, in % by mass, less than or equal to 0.4% of iron, less than or equal to 0.25% of silicon, and the balance being aluminum and unavoidable impurities.

13: The force measurement platform of claim 1, wherein the surface plate is made of stainless steel, brass, heavy duty polycarbonate plastic, and ultra-high molecular weight polyethylene plastic.

14: The force measurement platform of claim 1, wherein the base plate is made of stainless steel, brass, heavy duty polycarbonate plastic, and ultra-high molecular weight polyethylene plastic.

15: The force measurement platform of claim 1, meets the ANSI B157.1 standards.

16: The force measurement platform of claim 1, has a load capacity of up to 10 kilonewtons (kN).

17: A method of determining and monitoring postural stability, the method comprising:
calibrating the force measurement platform of claim 1;
applying a load to the surface of the force measurement assembly via at least one portion of a body and running the force measurement platform for 10 to 30 seconds to calculate a COP and record a sway; and
comparing the results of COP and sway against a reference database to determine and monitor postural stability.

18: The method of claim 17, is conducted in a state selected from the group consisting of eyes open, and eyes closed.

19: The method of claim 17, wherein the calibrating comprising:
removing any loads on the force measurement assembly and zeroizing the readings of the platform;
placing a known mass on the force measurement assembly, running the platform, and recording a displayed mass; and
calculating a calibration factor via a mathematical formula and setting the calibration factor in the data acquisition and processing unit.

20: The method of claim 19, wherein the mathematical formula (I) is:
$$Calibration factor [ I ] = \frac{Capacity ( g )}{Sensitivity ( \frac{mV}{V} ) \times Excitation Voltage ( V )}$$
